(19) United States
(12) Patent Application Publication
(10) Pub. No.: US 2003/0119063 A1
(43) Pub. Date: Jun. 26, 2003

(54) High accuracy protein identification

(76) Inventor: Thang T. Pham, Mountain View, CA (US)

Correspondence Address: Jonathan Alan Quine, Quine Intellectual Property Law Group, PO Box 458, Alameda, CA, 94501, US

(21) Appl. No.: 10/220,669

(22) PCT Filed: Mar. 18, 2002
(86) PCT No.: PCT/US02/08450

(51) Int. Cl. G01N 33/53; C12Q 1/37

(52) U.S. Cl. 435/7.1; 435/23

(57) ABSTRACT

The invention provides for the identification of target proteins in a sample based upon multiple sets of peptide fragment mass data obtained from the sample via gas phase ion spectroscopy. The sets of data are the product of analytical conditions that typically differ for each set such that cumulatively the data sets have higher information content than any individual set, thus enhancing the confidence level for accurate target protein identification. Probes, systems, and kits are additionally provided.

### CROSS-REFERENCES TO RELATED APPLICATIONS

Pursuant to 35 U.S.C. §§ 119 and/or 120, and any other applicable statute or rule, this application claims the benefit of and priority to U.S. Provisional Application No. 60/277,677, filed on Mar. 20, 2001, the disclosure of which is incorporated by reference.

### COPYRIGHT NOTIFICATION

Pursuant to 37 C.F.R. § 1.71(e), Applicants note that a portion of this disclosure contains material which is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### STATEMENT AS TO RIGHT TO INVENTIONS MADE UNDER FEDERALLY SPONSORED RESEARCH AND DEVELOPMENT

Not Applicable.

### BACKGROUND OF THE INVENTION

The human proteome includes numerous different proteins based on estimated gene numbers and considering additional complexity attributable to post-translational modification, degradation, and other cellular processes. The science of proteomics relates to the detection and identification of proteins, such as these from the human proteome. In particular, proteometric analyses are significant tools for drug discovery and development, which integrate genomics, mRNA analysis, and protein expression. See, Blackstock and Weir (1999) “Proteomics: quantitative and physical mapping of cellular proteins,” Trends Biotechnol. 17:121-127. For example, information obtained from proteome analysis can facilitate the identification of therapeutic targets and biomarkers that relate to the initiation and progression of a given pathological condition. Further, proteomics aids in the identification and elucidation of pharmacogenomic traits of key cellular proteins and in the design of optimized medications for individual patients. See, Evan and Relling (1999) “Pharmacogenomics: translating functional genomics into rational therapeutics,” Science 286:487-491.

Mass spectrometry (MS) is an analytical technique of increasing importance to proteomics and is often used in combination with other protein separation techniques, including one- and two-dimensional SDS-PAGE. In certain mass spectrometric approaches, proteins are identified based on detected peptide fragment mass profiles following digestion with a protease, such as trypsin, and a protein database query with the mass data. One problem associated with these approaches steins from impurities, such as non-target protein peptide fragments or proteins (e.g., keratins) or other biomolecules, which mask the detection of lower abundance or ‘low copy number’ target proteins. For example, keratin interference may originate from an inadequately purified protease. See, Zhang et al. (1998) “Purification of trypsin for mass spectrometric identification of proteins at high sensitivity,” Anal. Biochem., 261:124-127. These types of background chemical noises typically decrease protein identification confidence levels and can prevent accurate identification all together. Problems such as these are particularly pronounced for methods such as matrix-assisted laser desorption/ionization (MALDI) MS, which typically utilize complex samples for analysis.

Tandem mass spectrometry (MS/MS) is one method that has been used to reduce background chemical noise and thus, to improve the resolution of detected peptide fragment masses. This method involves coupling one mass spectrometer to a second. The first spectrometer serves to isolate the molecular ions of various components of a sample mixture, such as different proteins. It is typically equipped with a soft ionization source, such as a chemical ionization source, such that molecular ions or protonated ions are predominately generated. These ions are then introduced into the ionization source of the second mass spectrometer (e.g., a field-free collision chamber in which helium is passed), where they are fragmented to produce a series of mass spectra, one for each molecular ion produced in the first mass spectrometer. The chromatographic columns of gas chromatography/MS and liquid chromatography/MS serve the same function as the first spectrometer in MS/MS. However, the instrumentation for these devices is generally very expensive. See, Barker, Mass Spectrometry, 2^{nd} Ed., John Wiley & Sons, New York (1997).

From the above, it is apparent that techniques that inexpensively improve the information content of mass spectrometric analyses is highly desirable. The present invention provides new methods, and related systems, that improve the accuracy of mass spectrometric-based protein identification. These and a variety of additional features will become evident upon complete review of the following.

### SUMMARY OF THE INVENTION

The present invention generally relates to proteomics. In particular, the invention provides methods and related systems for identifying proteins in complex mixtures of biomolecules based upon detected peptide fragment masses. The methods generally include generating multiple peptide fragment mass profiles in which each profile is the product of a different condition. Peptide fragment masses are detected using gas phase ion spectrometric techniques, such as mass spectrometry. One advantage of the invention is that it dramatically increases the overall information content of gas phase ion spectrometric results.

In one aspect, the invention provides methods of producing at least one identity candidate for a target protein in a sample. Typically, the at least one identity candidate identifies the target protein. The methods include (a) fragmenting proteins in a first sample that includes the target protein to produce a fragmented sample that includes two or more peptide fragments of the target protein and (b) profiling peptide fragment masses in the fragmented sample by gas phase ion spectrometry under at least two different conditions. A first condition includes analyzing a first aliquot of the fragmented sample by the gas phase ion spectrometry to produce a first set of peptide fragment mass data. A second condition includes fractionating biomolecules in a second aliquot of the fragmented sample by a first fractionation technique to produce at least one sub-sample that includes a peptide fragment of the target protein, and analyzing one or more sub-samples by the gas phase ion spectrometry to produce a second set of peptide fragment mass data. Optionally, the method includes profiling peptide fragment masses in the fragmented sample under more than two different conditions, e.g., to provide additional sets of peptide fragment mass data. The gas phase ion spectrometry generally comprises mass spectrometry. In preferred embodiments, the mass spectrometry is laser desorption/ionization mass spectrometry. Optionally, the laser desorption/ionization mass spectrometry is surface enhanced (i.e., SELDI), matrix-assisted (i.e., MALDI), or the like. The methods also include (c) querying a database to produce the at least one identity candidate for the target protein based upon the first and second sets of peptide fragment mass data.

In preferred embodiments, the method further includes fractionating biomolecules in an initial sample by one or more second fractionation techniques to collect an initial sample fraction that includes the target protein in which the initial sample fraction is used as the first sample in (a). For example, the biomolecules in the initial sample are optionally fractionated by: (i) separating the biomolecules in the initial sample into a one- or two-dimensional array of spots in which each spot includes one or more of the biomolecules, and (ii) selecting and removing a spot from the array which is suspected of comprising the target protein. The first or second fractionation techniques are optionally independently selected from, e.g., electrophoresis, dialysis, filtration, centrifugation, or the like. As additional options, the first or second fractionation techniques are independently selected from, e.g., affinity chromatography, high performance liquid chromatography, ion exchange chromatography, size exclusion chromatography, or the like.

In one embodiment of the invention, gas phase ion spectrometric analysis of the first aliquot includes (i) contacting the first aliquot with at least one adsorbent bound to a surface of a probe which is removably insertable into a gas phase ion spectrometer, and (ii) desorbing and ionizing peptide fragments in the first aliquot from the probe and detecting the desorbed/ionized peptide fragments with the gas phase ion spectrometer to provide the first set of peptide fragment mass data. In another embodiment, gas phase ion spectrometric analysis of the first aliquot includes (i) contacting the first aliquot with a support-bound adsorbent (e.g., a bead or resin derivatized with an adsorbent or the like), (ii) placing the support-bound adsorbent on a probe in which the probe is removability insertable into a gas phase ion spectrometer, and (iii) desorbing and ionizing peptide fragments in the first aliquot from the probe and detecting the desorbed/ionized peptide fragments with the gas phase ion spectrometer to provide the first set of peptide fragment mass data.

In some embodiments, gas phase ion spectrometric analysis of the one or more sub-samples of the second aliquot includes (i) contacting the second aliquot with the adsorbent bound to a surface of al probe which is removably insertable into a gas phase ion spectrometer in which the adsorbent captures one or more peptide fragments from the target protein. This embodiment also includes (ii) removing non-captured material from the probe in which the one or more captured peptide fragments include a first sub-sample of the second aliquot, and (iii) desorbing and ionizing the one or more captured peptide fragments from the probe and detecting the one or more desorbed/ionized peptide fragments with the gas phase ion spectrometer to provide the second set of peptide fragment mass data. In other embodiments of the invention, gas phase ion spectrometric analysis of the one or mole sub-samples of the second aliquot includes (i) contacting the second aliquot with a support-bound adsorbent (e.g., a bead or resin derivatized with an adsorbent or the like in which the support-bound adsorbent captures one or more peptide fragments from the target protein, and (ii) removing non-captured material from the support-bound adsorbent in which the one or more captured peptide fragments on the support-bound adsorbent include a first sub-sample of the second aliquot. This embodiment also includes (iii) placing the support-bound adsorbent on a probe in which the probe is removably insertable into a gas phase ion spectrometer, and (iv) desorbing and ionizing the one or more captured peptide fragments from the probe and detecting the one or more desorbed/ionized peptide fragments with the gas phase ion spectrometer to provide the second set of peptide fragment mass data. Non-captured material is generally removed by one or more washes. For example, each of the one or more washes optionally includes an identical or a different elution condition relative to at least one preceding wash. Elution conditions typically differ according to, e.g., pH, buffering capacity, ionic strength, a water structure characteristic, detergent type, detergent strength, hydrophobicity, dielectric constant, concentration of at least one solute, or the like.

The adsorbents utilized in the methods of the present invention include various alternative embodiments. For example, in certain embodiments the adsorbent includes a chromatographic adsorbent. Suitable chromatographic adsorbents include, e.g., an electrostatic adsorbent, a hydrophobic interaction adsorbent, a hydrophilic interaction adsorbent, a salt-promoted interaction adsorbent, a reversible covalent interaction adsorbent, a coordinate covalent interaction adsorbent, or the like. In other embodiments, the adsorbent is a biomolecular interaction adsorbent, such as an affinity adsorbent, a polypeptide, an enzyme, a receptor, an antibody, or the like. The biomolecular interaction adsorbent generally specifically captures at least one peptide fragment from the target protein. In certain embodiments the adsorbent includes a polypeptide that specifically binds an immunoglobulin and the method comprises exposing the first or second aliquot to the immunoglobulin in which the immunoglobulin specifically binds the one or more peptide fragments from the target protein to form a peptide fragment-complex, and contacting the peptide fragment-complex to the adsorbent.

The probe generally includes a substrate with at least one surface feature that includes the absorbent bound to the substrate, or capable of including the support-bound adsorbent. The substrate typically includes one or more of, e.g., glass, ceramic, plastic, a magnetic material, a polymer, an organic polymer, a conductive polymer, a native biopolymer, a metal, a metalloid, an alloy, a metal coated with an organic polymer, or the like. The at least one surface feature typically includes a plurality of surface features. For example, the plurality of surface features is optionally arranged in a line, an orthogonal array, a circle, an n-sided polygon, wherein n is three or greater, or the like. As a further example, the plurality of surface features includes a logical or spatial array. In certain embodiment, each of the plurality of surface features includes identical or different absorbents, or one or more combinations thereof. In other embodiments, at least two of the plurality of surface features include identical or different adsorbents, or one or more combinations thereof.

Optionally, the method further includes generating a table of masses for peptide fragments in the first and second sets of peptide fragment mass data prior to (c). The method typically includes comparing amounts of peptide fragments detected in the first or second sets of peptide fragment mass data with one or more controls (e.g., to calibrate the detection system of the gas phase ion spectrometer). In addition, individual peptide fragments in the first or second sets of peptide fragment mass data are optionally quantified. The method also optionally includes producing identity candidates for multiple target proteins in the first sample (e.g., for protein expression profiling or the like). In some embodiments, the identity candidate for the target protein aids in the diagnosis of pathological conditions.

In preferred embodiments, the first and second sets of peptide fragment mass data are in a computer-readable form. For example, (c) generally includes operating a programmable computer and executing an algorithm that determines closeness-of-fit between the computer-readable data and database entries, which entries correspond to masses of identified proteins or peptide fragments thereform to produce the at least one identity candidate for the target protein based upon one or more detected peptide fragment masses in the first and second sets of peptide fragment mass data. In some embodiments, the algorithm includes an artificial intelligence algorithm or a heuristic learning algorithm. For example, the artificial intelligence algorithm optionally includes one or more of, e.g., a fuzzy logic instruction set, a cluster analysis instruction set, a neural network, a genetic algorithm, or the like.

The present invention also includes a method of producing at least one identity candidate for a target protein that includes (a) fragmenting proteins in a first sample that includes the target protein with one or more enzymes to produce a fragmented sample that includes two or more peptide fragments of the target protein, and (b) profiling peptide fragment masses in the fragmented sample by gas phase ion spectrometry under at least two different conditions. A first condition generally includes analyzing a first aliquot of the fragmented sample by the gas phase ion spectrometry to produce a first set of peptide fragment mass data. A second condition includes fractionating biomolecules in a second aliquot of the fragmented sample by at least one first fractionation technique to produce at least one sub-sample that includes a peptide fragment of the target protein, and analyzing one or more sub-samples by the gas phase ion spectrometry to produce at least a second set of peptide fragment mass data. The method also includes (c) querying at least one database to produce the at least one identity candidate for the target protein based upon the first and second sets of peptide fragment mass data.

The invention also relates to a method of producing at least one identity candidate for a target protein that includes (a) fragmenting proteins in a first sample that includes the target protein with trypsin to produce a fragmented sample that includes two or more peptide fragments of the target protein, and (b) profiling peptide fragment masses in the fragmented sample by surface enhanced desorption/ionization time-of-flight mass spectrometry under at least two different conditions. A first condition typically includes analyzing a first aliquot of the fragmented sample by the surface enhanced desorption/ionization time-of-flight mass spectrometry to produce a first set of peptide fragment mass data. A second condition generally includes fractionating biomolecules in a second aliquot of the fragmented sample into two or more sub-samples by affinity chromatography to produce at least one sub-sample that includes a peptide fragment of the target protein, and analyzing one or more sub-samples by the surface enhanced depositor/ionization time-of-flight mass spectrometry to produce it least a second set of peptide fragment mass data. The method additionally includes (c) querying at least one database to produce the at least one identity candidate for the target protein based upon the first and second sets of peptide fragment mass data.

The present invention also provides a system capable of producing at least one identity candidate for a target protein in a sample. The system includes (a) one or more absorbents capable of capturing peptide fragments in the sample under at least two different conditions, and (b) a gas phase ion spectrometer (e.g., a mass spectrometer, such as a laser desorption/ionization mass spectrometer) able to profile masses of peptide fragments captured by the one or more adsorbents under the at least two different conditions to provide at least two sets of peptide fragment mass data, each set corresponding to peptide fragments detected under a different condition. The system also includes (c) a processor, operably connected to the gas phase ion spectrometer, that includes at least one computer, program providing logic instructions capable of determining closeness-of-fit between one or more detected peptide fragment masses in the sets of peptide fragment mass data and database entries, which entries correspond to masses of identified proteins or peptide fragments therefrom to produce the at least one identity candidate for the target protein based upon the one or more detected peptide fragment masses. A computer or other logic device typically includes the processor and in certain embodiments, the computer is external to the gas phase ion spectrometer. In other embodiments, the gas phase ion spectrometer includes the processor (e.g., the processor is typically a component of the computer). The adsorbents generally include solid phase adsorbents, which are optionally provided as a probe that includes a substrate with at least one surface feature that includes the solid phase adsorbents bound to the substrate. The probe is typically removably insertable into the gas phase ion spectrometer. In other embodiments, the solid phase adsorbents include beads or resins derivatized with the adsorbents. For example, the beads or resins derivatized with the absorbents are generally suitable for being placed on a probe removably insertable into the gas phase ion spectrometer.

### BRIEF DESCRIPTION OF THE DRAWINGS

FIG. 1 schematically shows a surface enhanced laser desorption/ionization assay of an unfractionated first aliquot of a fragmented sample.

FIG. 2 schematically illustrates a surface enhanced laser desorption/ionization assay of a second or subsequent aliquot of a fragmented sample.

FIG. 3 is a flow chart that schematically shows steps involved in an embodiment of the invention for identifying a target protein based on two sets of peptide fragment mass data.

FIG. 4 is a flow chart that schematically illustrates steps involved in an embodiment of the invention for querying a protein database with multiple sets of peptide fragment mass data to identify a target protein.

FIG. 5 schematically depicts a surface enhanced laser desorption/ionization time-of-flight mass spectrometry system.

FIG. 6 is schematically illustrates a representative example information appliance or digital device in which various aspects of the present invention may be embodied.

FIGS. 7A-E are mass spectral traces between 900 and 6000 Daltons showing detected peptide fragments from a tryptic digest of bovine transferrin under different conditions.

FIGS. 8A-E are mass spectral traces between 900 and 2500 Daltons showing detected peptide fragments from a tryptic digest of bovine transferrin under different conditions.

FIGS. 9A-E are mass spectral traces between 2500 and 6000 Daltons showing detected peptide fragments from a tryptic digest of bovine transferrin under different conditions.

FIGS. 10A-E are mass spectral traces between 900 and 5000 Daltons showing peptide maps of a tryptic digest of bovine transferrin under different conditions.

FIG. 11 shows a display screen for a ProFound database search using a peptide map generated by MALDI.

FIG. 12 shows a display screen for a ProFound database search showing an analysis of the best candidate using MALDI data.

FIG. 13 shows a display screen for a ProFound database search using a peptide map generated by SELDI.

FIG. 14 shows a display screen for a ProFound database search showing an analysis of the best candidate using SELDI data.

FIG. 15 shows a display screen for a MASCOT database search using a peptide map generated by MALDI.

FIG. 16 shows a display screen for a MASCOT database search showing an analysis of the best candidate using MALDI data.

FIG. 17 shows a display screen for a MASCOT database search using a peptide map generated by SELDI.

FIG. 18 shows a display screen for a MASCOT database search showing an analysis of the best candidate using SELDI data.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd Ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

“Substrate” or “probe substrate” refers to a solid phase onto which an adsorbent can be provided (e.g., by attachment, deposition, or the like). “Surface feature” refers to a particular portion, section, or area of a substrate or probe substrate onto which adsorbent can be provided.

“Surface” refers to the exterior or upper boundary of a body or a substrate.

“Plate” refers to a thin piece of material that is substantially flat or planar, and it can be in any suitable shape (e.g., rectangular, square, oblong, circular, etc.).

“Substantially flat” refers to a substrate having the major surfaces essentially parallel and distinctly greater than the minor surfaces (e.g., a strip or a plate).

“Adsorbent” refers to any material capable of adsorbing an analyte (e.g., a peptide fragment). The term “adsorbent” is used herein to refer both to a single material (“monoplex adsorbent”) (e.g., a compound or functional group) to which the analyte is exposed, and to a plurality of different materials (“multiplex adsorbent”) to which the analyte is exposed. The adsorbent materials in a multiplex adsorbent are referred to as “adsorbent species.” For example, a surface feature on a probe substrate can comprise a multiplex absorbent characterized by many different adsorbent species (e.g., ion exchange materials, metal chelators, antibodies, or the like), having different binding characteristics. Substrate material itself can also contribute to adsorbing an analyte and may be considered part of all “adsorbent.” A “biomolecular interaction adsorbent” or “biospecific adsorbent,” such as an affinity adsorbent, a polypeptide, an enzyme, a receptor, an antibody (e.g., a monoclonal antibody, etc.), or the like, typically has higher specificity for a target analyte than a “chromatographic adsorbent,” which includes, e.g., an anionic adsorbent, a cationic adsorbent, a hydrophobic interaction adsorbent, a hydrophilic interaction adsorbent, a metal-chelating adsorbent, or the like.

“Adsorption,” “capture,” or “retention” refers to the detectable binding between an adsorbent and an analyte (e.g., a peptide fragment) either before or after washing with an eluant (selectivity threshold modifier) or a washing solution.

“Eluant,” “wash,” or “washing solution” refers to an agent that can be used to mediate adsorption of all analyte to an absorbent. Eluants and washing solutions also are referred to as “selectivity threshold modifiers.” Eluants and washing solutions can be used to wash and remove unbound or non-captured materials from the probe substrate surface.

“Specific binding” refers to binding that is mediated primarily by the basis of attraction of all adsorbent for a designated analyte (e.g., a peptide fragment from a target protein). For example, the basis of attraction of an anionic exchange adsorbent for an analyte is the electrostatic attraction between positive and negative charges. Therefore, anionic exchange adsorbents engage in specific binding with negatively charged species. The basis for attraction of a hydrophilic adsorbent for an analyte is hydrogen bonding. Therefore, hydrophilic adsorbents engage in specific binding with electrically polar species or the like.

“Resolve,” “resolution,” or “resolution of analyte” refers to the detection of at least one analyte in a sample. Resolution includes the detection and differentiation of a plurality of analytes in a sample by separation and subsequent differential detection. Resolution does not require the complete separation of an analyte from all other analytes in a mixture. Rather, any separation that allows the distinction between at least two analytes suffices.

“Probe” refers to a device that, when positionally engaged in an interrogatable relationship to an ionization source, e.g., a laser desorption/ionization source, and in concurrent communication at atmospheric or subatmospheric pressure with a detector of a gas phase ion spectrometer, can be used to introduce ions derived from an analyte into the spectrometer. As used herein, the “probe” is typically reversibly engageable (e.g., removably insertable) with a probe interface that positions the probe in an interrogatable relationship with the ionization source and in communication with the detector. A probe will generally comprise a substrate comprising a sample presenting surface on which an analyte is presented to the ionization source. “Ionization source” refers to a device that directs ionizing energy to a sample presenting surface of a probe to desorb and ionize analytes from the probe surface into the gas phase. The preferred ionization source is a laser (used in laser desorption/ionization), in particular, nitrogen lasers, Nd—Yag lasers and other pulsed laser sources. Other ionization sources include fast atoms (used in fast atom bombardment), plasma energy (used in plasma desorption) and primary ions generating secondary ions (used in secondary ion mass spectrometry).

“Gas phase ion spectrometer” refers to an apparatus that detects gas phase ions. In the context of this invention, gas phase ion spectrometers include an ionization source used to generate the gas phase ions. Gas phase ion spectrometers include, for example, mass spectrometers, ion mobility spectrometers, and total ion current measuring devices.

“Gas phase ion spectrometry” refers to a method comprising employing an ionization source to generate gas phase ions from an analyte presented on a sample presenting surface of a probe and detecting the gas phase ions with a gas phase ion spectrometer.

“Mass spectrometer” refers to a gas phase ion spectrometer that measures a parameter which can be translated into mass-to-charge ratios of gas phase ions. Mass spectrometers generally include an inlet system, an ionization source, an ion optic assembly, a mass analyzer, and a detector. Examples of mass spectrometers are time-of-flight, magnetic sector, quadrapole filter, ion trap, ion cyclotron resonance, electrostatic sector analyzer and hybrids of these.

“Mass spectrometry” refers to a method comprising employing an ionization source to generate gas phase ions from an analyte presented on a sample presenting surface of a probe and detecting the gas phase ions with a mass spectrometer.

“Laser desorption mass spectrometer” refers to a mass spectrometer which uses laser as a means to desorb, volatilize, and ionize an analyte.

“Desorption ionization” refers to generating ions by desorbing them from a solid or liquid sample with a high-energy particle beam (e.g., a laser). Desorption ionization encompasses various techniques including, e.g., surface enhanced laser desorption, matrix-assisted laser desorption, fast atom bombardment, plasma desorption, or the like.

“Matrix-assisted laser desorption/ionization” or “MALDI” refers to an ionization source that generates ions by desorbing them from a solid matrix material with a pulsed laser beam.

“Detect” refers to identifying the presence, absence or amount of the object to be detected.

“Biomolecule” or “bioorganic molecule” refers to an organic molecule typically made by living organisms. This includes, for example, molecules comprising nucleotides, amino acids, sugars, fatty acids, steroids, nucleic acids, polypeptides, peptides, peptide fragments, carbohydrates, lipids, and combinations of these (e.g., glycoproteinis, ribonucleoproteins, lipoproteins, or the like).

“Biological material” refers to any material derived from an organism, organ, tissue, cell or Virus. This includes biological fluids such as saliva, blood, urine, lymphatic fluid, prostatic or seminal fluid, milk, etc., as well as extracts of any of these, e.g., cell extracts, cell culture media, fractionated samples, or the like.

“Energy absorbing molecule” or “EAM” refers to a molecule that absorbs energy from an ionization source in a mass spectrometer thereby enabling desorption of analyte, such as a peptide fragment, from a probe surface. Depending on the size and nature of the analyte, the energy absorbing molecule can optionally be used. Energy absorbing molecules used in MALDI are frequently referred to as “matrix.” Cininamic acid derivatives, sinapinic acid (“SPA”), cyano hydroxy cinnamic acid (“CHCA”), and dihydroxybenzoic acid are frequently used as energy absorbing molecules in laser desorption of bioorganic molecules. See, U.S. Pat. No. 5,719,060 to Hutchens and Yip for additional description of energy absorbing molecules.

The terms “polypeptide,” “peptide,” and “protein” are used interchangeably herein to refer to a polymer of amino acid residues. The terms apply to amino acid polymers in which one or more amino acid residues are analogs, derivatives or mimetics of corresponding naturally occurring amino acids,,as well as to naturally Occurring amino acid polymers. For example, polypeptides can be modified or derivatized, e.g., by the addition of carbohydrate residues to form glycoproteins. The terms “polypeptide,” “peptide,” and “protein” include glycoproteins, as well as non-glycoproteins.

A “target protein” refers to a protein to be identified.

“Fragmentation,” “digestion,” or “cleavage” refers to a process that occurs when enough energy is concentrated in a bond, causing the vibrating atoms to move apart beyond a bonding distance. For example, target proteins are enzymatically, chemically, or physically fragmented prior to detection.

A “peptide fragment” refers to a subsequence of amino acids derived from a polypeptide, peptide, or protein upon fragmentation of the polypeptide, peptide, or protein.

An “identity candidate” refers to a database entry corresponding to a known polypeptide, peptide, or protein that matches, corresponds to, or comprises a peptide fragment, set of peptide fragments, or one or more character strings corresponding thereto, derived from a target protein. Identity candidates produced by a database query are typically ranked according to probability of matching, corresponding to, or comprising a peptide fragment, or set of peptide fragments, derived from a target protein.

A “set” refers to a collection of at least two molecules. For example, a set typically includes between about two and about 10⁶ molecules, more typically includes between about 100 and about 10⁵ molecules, and usually includes between about 1000 and about 10⁴ molecules.

“Derivative” refers to a chemical substance related structurally to another substance, or a chemical substance that can be made from another substance (i.e., the substance it is derived from), e.g., through chemical or enzymatic modification.

“Antibody” refers to a polypeptide ligand substantially encoded by all immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well characterized fragments produced by digestion with various peptidases. This includes, e.g., Fab′ and F(ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. The “Fc” portion of an antibody refers to that portion of an immunoglobulin heavy chain that comprises one or more heavy chain constant region domains, CH1, CH2 and CH3, but does not include the heavy chain variable region.

“Immunoassay” is an assay that uses an antibody to specifically bind an antigen (e.g., a peptide fragment). The immunoassay is characterized by the use of specific binding properties of a particular antibody to isolate, target, and/or quantify the antigen.

### DETAILED DISCUSSION OF THE INVENTION INTRODUCTION

Significant technological advances in protein chemistry in the last two decades have established mass spectrometry as an indispensable tool for protein study (Carr et al., (1991) “Integration of mass spectrometry in analytical biotechnology,” Anal. Chem. 63(24):2802-2824; Carr et al., “Overview of Peptide and Protein Analysis by Mass Spectrometry,” Current Protocols in Molecular Biology, John Wiley & Sons, Inc., New York, unit 10.21, pp. 10.21.1-10.21.27 (1998); Patterson, “Protein Identification and Characterization by Mass Spectrometry,” Current Protocols in Molecular Biology, John Wiley & Sons, Inc., New York, unit 10.22, pp. 10.22.1-10.22.24 (1998); Bakhtiar and Tsc (2000) “Biological Mass Spectrometry: A Primer,” Mutagenesis 15:415-430; and Siuzdak, Mass Spectrometry for Biotechnology, Academic Press, San Diego (1996)). Although the resolving power of many chromatographic- and electrophoretic-based separations remains analytically useful, the high sensitivity, speed, and reproducibility of mass spectrometry have boosted its application in all aspects of proteome analysis, including discovery, identification (e.g., peptide mapping, sequencing, etc.), quantification, and structural characterization.

Analogous to the oligonucleotide chip technologies that allow the study of gene expression profiles, protein biochip technologies have been developed in which proteins are captured on surface features of probes for analysis by mass spectrometry. One such technology takes advantage of surface enhanced laser desorption/ionization time-of-flight mass spectrometry to facilitate protein profiling of complex biologic mixtures. In a version of this technology, affinity mass spectrometry, substrate-bound affinity reagents, either chromatographic or biospecific, capture analytes from a sample. The captured analytes are then desorbed/ionized from the substrate and detected by mass spectrometry. (See, e.g., Hutchens and Yip (1993) “New desorption strategies for the mass spectrometric analysis of macromolecules,” Rapid Commun. Mass Spectrom. 7:576-580, Kuwata et al., (1998) “Bactericidal domain of lactoferrin: detection, quantitation, and characterization of lactoferricin in serum by SELDI Affinity Mass Spectrometry,” Bioch. Bioph. Res. Comm. 245:761-773, U.S. Pat. No. 5,719,060 to Hutchens and Yip, and WO 98/59360 (Hutchens and Yip)). This innovative technology has numerous advantages over other techniques, such as 2D-PAGE. For example, it is much faster, has higher throughput, requires orders of magnitude lower amounts of sample, has a sensitivity for detecting analyte in the picomole to attomole range, can effectively resolve proteins, peptide fragments, and other materials having m asses in the range of about 2 kDa to about 20 kDa, and is directly applicable to clinical assay development.

The present invention provides methods of accurately identifying target proteins (or of at least providing identity candidates for a given target protein) in a sample. The methods generally include fragmenting proteins in a sample that includes a target protein to produce two or more peptide fragments from the target protein, and profiling peptide fragment masses in the sample by gas phase ion spectrometry under at least two different conditions. One condition includes analyzing a first aliquot of the sample by gas phase ion spectrometry to produce one set of peptide fragment mass data in which all peptide fragments in the sample are represented and at least theoretically visible in the mass spectral trace. Other conditions include fractionating biomolecules in at least a second aliquot of the sample (e.g., by retentate chromatography, affinity chromatography and/or by other fractionation techniques) to produce sub-samples that include one or more peptide fragments of the target protein, and analyzing the sub-samples by gas phase ion spectrometry to produce additional sets of peptide fragment mass data. The additional sets of peptide fragment mass data typically include reduced levels of background chemical noise relative to the spectrum generated from the first aliquot. Reduced background noise generally leads to improved resolution of particular peptide fragments from the target protein. Thereafter, the methods include querying a protein database to identify the target protein (or to produce identity candidates therefore) based upon all of the sets of peptide fragment mass data. Since these methods typically provide greater numbers of peptide fragments to the database query than, if only a single set of mass data were used, the confidence level of accurately identifying the target protein is greatly increased. In addition, the invention also includes biochips, kits, and systems.

I. Sample Preparation Prior to Fragmentation

The methods of this invention begin with a sample provided for analysis that comprises the target protein. This sample may be used directly, or may be prepared for analysis by, for example, fractionation of the sample to produce a sub-sample comprising the target protein.

A. Target Protein Sources

The samples used in this invention are optionally derived from any biological material source. This includes body fluids such as blood, serum, saliva, urine, prostatic fluid, seminal fluid, seminal plasma, lymph, lung/bronchial washes, mucus, feces, nipple secretions, sputum, tears, or the like. It also includes extracts from biological samples, such as cell lysates, cell culture media, or the like. For example, cell lysate samples are optionally derived from, e.g., primary tissue or cells, cultured tissue or cells, normal tissue or cells, diseased tissue or cells, benign tissue or cells, cancerous tissue or cells, salivary glandular tissue or cells, intestinal tissue or cells, neural tissue or cells, renal tissue or cells, lymphatic tissue or cells, bladder tissue or cells, prostatic tissue or cells, urogenital tissues or cells, tumoral tissue or cells, tumoral neovasculature tissue or cells, or the like. The specific exemplary target protein sources listed herein are offered to illustrate but not to limit the present invention Additional sources of protein samples are known in the art and are readily obtainable.

Biological samples are optionally collected according to any known technique, such as venipuncture, biopsy, or the like. Many references are available for the culture and production of many cells, including cells of bacterial, plant, animal (especially mammalian) and archebacterial origin: See e.g., Ausubel et al., eds., Current Protocols in Molecular Biology, a joint venture between Greene Publishing Associates, Inc. and John Wiley & Sons, Inc., New York (supplemented through 1999), Berger and Kimmel, Guide to Molecular Cloning Techniques, Methods in Enzymology volume 152 Academic Press, Inc., San Diego, Calif., Sambrook et al., Molecular Cloning—A Laboratory Manual (2nd Ed.), Vol. 1-3, Cold Spring Harbor Laboratory, Cold Spring Harbor, N.Y. (1989), Freshney, Culture of Animal Cells, a Manual of Basic Techniques, 3^{rd} Ed., Wiley-Liss, New York (1994); and Humason, Animal Tissue Techniques, 4^{th} Ed., W. H. Freeman and Company, New York (1979), Doyle and Griffith, Mammalian Cell Culture: Essential Techniques, John Wiley and Sons, New York (1997), Ricciardelli, et al. (1989) In vitro Cell Dev. Biol. 25:1016-1024, and the references cited therein. Plant cell culture is described in, e.g., Payne et al., Plant Cell and Tissue Culture in liquid System, John Wiley & Sons, Inc., New York (1992), Gamborg and Phillips (Eds) Plant Cell, Tissue and Organ Culture; Fundamental Methods Springer Lab Manual, Springer-Verlag, New York (1995), and the references cited therein. Cell culture media in general are set forth in Atlas and Parks (Eds), The Handbook of Microbiological Media, CRC Press, Boca Raton (1993). Additional information for cell culture is found in available commercial literature such as the Life Science Research Cell Culture Catalogue (1998) from Sigma-Aldrich, Inc (St Louis, Mo.) (“Sigma-LSRCCC”) and, e.g., the Plant Culture Catalogue and supplement (1997) also from Sigma-Aldrich, Inc (St Louis, Mo.) (“Sigma-PCCS”).

Polypeptides of the invention are optionally recovered and purified from cell cultures by any of a number of methods well known in the art, including ammonium sulfate or ethanol precipitation, acid extraction, anion or cation exchange chromatography, phosphocellulose chromatography, hydrophobic interaction chromatography, affinity chromatography (e.g., using any of the tagging systems noted herein), hydroxylapatite chromatography, and lectin chromatography. Preferably, the sample is in a liquid form from which solid materials have been removed. In addition to the references noted herein, a variety of purification methods are well known in the art, including, e.g., those set forth in Sandana, Bioseparation of Proteins, Academic Press, Inc., San Diego (1997), Bollag et al., Protein Methods, 2^{nd} Ed., Wiley-Liss, New York (1996), Walker, The Protein Protocols Handbook, Humana Press, New Jersey (1996), Harris and Angal, Protein Purification Applications: A Practical Approach, IRL Press, Oxford (1990), Harris and Angal (Ed), Protein Purification Methods: A Practical Approach, IRL Press, Oxford (1989), Scopes, Protein Purification: Principles and Practice, 3^{rd} Ed., Springer Verlag, New York (1993), Janson and Ryden, Protein Purification: Principles, High Resolution Methods and Applications, 2nd Ed., Wiley-VCH, New York (1998), Walker, Protein Protocols on CD-ROM, Humana Press, New Jersey (1998), and the references cited therein. Sample fractionation techniques are described further below.

B. Biomolecule Fractionation

While an initial sample comprising the target protein can be analyzed directly, in preferred embodiments, the methods include fractionating biomolecules in an initial sample by one or a combination of fractionation techniques described below or otherwise known in the art to be useful for separating biomolecules to collect a sample fraction that includes the target protein prior to mass profiling. Fractionation is typically utilized to decrease the complexity of analytes in the sample to assist detection and characteristic of peptide fragments from a target protein or proteins. Moreover, fractionation protocols can provide additional information regarding physical and chemical characteristics of target proteins. For example, if a sample is fractionated using an anion-exchange spin column, and if a target protein is eluted at a certain pH, this elution characteristic provides information regarding binding properties of the target protein. In another example, a sample can be fractionated to remove proteins or other molecules in the sample that are present in a high quantity and/or which would otherwise interfere with the detection of a particular target protein.

Suitable sample fractionation protocols will be apparent to one of skill in the art. Exemplary fractionation techniques optionally utilized with the methods described herein include those based on size, such as size exclusion chromatography, gel electrophoresis, membrane dialysis, filtration, centrifugation (e.g., ultracentifugation), or the like. Separations are also optionally based on charges carried by analytes (e.g., as with anion or cation exchange chromatography), on analyte hydrophobicity (e.g., as with C₁-C₁₈ resins), on analyte affinity (e.g., as with immunoaffinity, immobilized metals, or dyes), or the like. In preferred embodiments, fractionation is effected using high performance liquid chromatography (HPLC). Other methods of fractionation include, e.g., crystallization and precipitation. In certain embodiments, following initial sample fractionation, the target protein comprises at least about 50% by weight of total protein in, e.g., the first sample, whereas in others the target protein comprises at least about 50% of the total protein molecules in, e.g., the first sample. Many of these fractionation techniques are described further in, e.g., Walker (Ed.) Basic Protein and Peptide Protocols: Methods in Molecular Biology (1994), Vol. 32, The Humana Press, Totowa, N.J., Fallon et al. (Eds.) Applications of HPLC in Biochemistry: Laboratory Techniques in Biochemistry and Molecular Biology (1987), Elsevier Science Publishers, Amsterdam, Matejtschuk (Ed.) Affinity Separations: A Practical Approach (1997), IRL Press, Oxford, Scouten, Affinity Chromatography: Bioselective Adsorption on Inert Matrices (1981) John Wiley & Sons, New York, Hydrophobic Interaction Chromatography: Principles and Methods (1993) Pharmacia, Brown, Advances in Chromatography (1998) Marcel Dekker, Inc., New York; Lough and Wainer (Eds.), High Performance Liquid Chromatography: Fundamental Principles and Practice (1996) Blackie Academic and Professional, London, Mant and Hodges (Eds.), High Performance Liquid Chromatography of Peptides and Proteins. Separation, Analysis and Conformation (1991) CRC Press, Boca Raton, Weiss, Ion Chromatography, 2^{nd} ed. (1995) VCH, New York, Ion-Exchange Chromatography: Principles and Methods (1991) Pharmacia, Smith, The Practice of Ion Chromatography (1990) Krieger Publishing Company, Melbourne, Fla., Bidlingmeyer, Practical HPLC Methodology and Applications (1992) John Wiley & Sons, Inc., New York, and Rickwood et al., Centrifugation: Essential Data Series (1994) Cold Spring Harbor Laboratory, New York. Certain of these techniques are illustrated further below.

1. Size Exclusion Chromatography

In one embodiment, a sample can be fractionated according to the size of, e.g., proteins in a sample using size exclusion chromatography. For a biological sample in which the amount of sample available is small, preferably a size selection spin column is used. For example, K-30 spin column (Ciphergen Biosystems, Inc.) can be used. In general, the first fraction that is eluted from the column (“fraction 1”) has the highest percentage of high molecular weight proteins; fraction 2 has a lower percentage of high molecular weight proteins; fraction 3 has even a lower percentage of high molecular weight proteins; fraction 4 has the lowest amount of large proteins; and so on. Each fraction is optionally then analyzed by gas phase ion spectrometry for the detection of particular proteins according to the methods described herein.

2. Separation of Biomolecules by Gel Electrophoresis

In another embodiment, biomolecules (e.g., proteins, nucleic acids, etc.) in a sample can be separated by high-resolution electrophoresis, e.g., one- or two-dimensional gel electrophoresis. Northern blotting, or the like. A fraction suspected of containing a target protein can be isolated and further analyzed by gas phase ion spectrometry as described herein. Preferably, two-dimensional gel electrophoresis is used to generate two-dimensional array of spots of biomolecules, including one or more target proteins. See, e.g., Jungblut and Thiede, Mass Spectr. Rev. 16:145-162(1997).

Two-dimensional gel electrophoresis is optionally performed using methods known in the art. See, e.g., Deutscher ed., Methods In Enzymology vol. 182. Typically, biomolecules in a sample are separated by, e.g., isoelectric focusing, during which biomolecules in a simple are separated in a pH gradient until they reach a spot where their net charge is zero (i.e., their isoelectric point). This first separation step results in a one-dimensional array of biomolecules. The biomolecules in the one dimensional airily are further separated using a technique generally distinct from that used in the first separation step. For example, in a second dimension, biomolecules separated by isoelectric focusing are further separated using a polyacrylamide gel, such as polyacrylamide gel electrophoresis in the presence of sodium dodecyl sulfate (SDS-PAGE). SDS-PAGE gel allows further separation based on molecular masses of biomolecules. Typically, two-dimensional gel electrophoresis can separate chemically different biomolecules in the molecular mass range from of from about 1000 to about 200,000 Da within complex mixtures.

Biomolecules in the two-dimensional array are optionally detected using any suitable method known in the art. For example, biomolecules in a gel can be labeled or stained (e.g., by Coomassie Blue, silver staining, fluorescent tagging, radioactive labeling, or the like). If gel electrophoresis generates spots that correspond to the molecular weight of one or more target proteins, the spot can be is further analyzed by gas phase ion spectrometry according to the methods of the invention. For example, spots can be excised from the gel and proteins in the selected spot can be cleaved or otherwise fragmented into smaller peptide fragments using, e.g., cleaving reagents, such as proteases (e.g., trypsin), prior to gas phase ion spectrometeric analysis. Alternatively, the gel containing biomolecules can be transferred to an inert membrane by applying an electric field. Then, a spot on the membrane that approximately corresponds to the molecular weight of a marker can be analyzed according to the methods described herein. In gas phase ion spectrometry, the spots can be analyzed using any suitable technique, such as MALDI or surface enhanced laser desorption/ionization (e.g., using ProteinChip® array) as described in detail below.

3. High Performance Liquid Chromatography

In yet another embodiment, high performance liquid chromatography (HPLC) can be used to separate a mixture of biomolecules in a sample based on their different physical properties, such as polarity, charge, size, or the like. HPLC instruments typically consist of a mobile phase reservoir, a pump, an injector, a separation column, and a detector. Biomolecules in a sample are separated by injecting an aliquot of the sample onto the column. Different biomolecules in the mixture pass through the column at different rates due to differences in their partitioning behavior between the mobile liquid phase and the stationary phase. A fraction that corresponds to the molecular weight and/or physical properties of, e.g., one or more target proteins can be collected. The fraction can then be analyzed by gas phase ion spectrometry following protein fragmentation according to the methods described herein to detect peptide fragments from target proteins. For example, the spots can be analyzed using either MALDI or surface enhanced laser desorption/ionization (e.g., using ProteinChip® array) as described in detail below.

II. Target Protein Fragmentation

Prior to profiling peptide fragment masses by gas phase ion spectroscopy, proteins in the samples of the invention are fragmented or digested. Fragmentation is optionally effected using any technique that produces peptide fragments from proteins in a sample. Many of these techniques are generally known in the art. For example, proteins are optionally fragmented enzymatically, chemically, or physically. Fragmentation is typically non-specific (i.e., random), specific (i.e., only at particular sites in a given protein), or selective (i.e., preferential). Physical fragmentation methods, such as physical shearing, thermal cleavage, or the like typically result in non-specific protein fragmentation. In contrast, enzymatic and chemical fragmentation methods may produce non-specifically or specifically cleaved peptide fragments from proteins in a sample. Examples, of chemical agents that result in specific cleavage include, cyanogen bromide (CNBr), which fragments polypeptide chains only on the carboxyl side of methionine residues, O-lodosobenxoate, which cleaves to the carboxyl side of tryptophan residues, hydroxylamine, which fragments peptide bonds between asparagine and glycine residues, and 2-nitro-5-thiocyanobenzoate, which cleaves to the amino side of cysteine residues. Other chemical agents that effect protein fragmentation, whether non-specific, selective, or specific, are known and optionally used in the methods of the present invention. Examples of enzymes that yield specifically or selectively cleaved peptide fragments, include trypsin (cleaves on the carboxyl side of arginine and lysine residues, clostripain (cleaves on the carboxyl side of arginine residues), chymotrypsin (cleaves preferentially on the carboxyl side of aromatic and certain other bulky nonpolar residues), and Staphylococcal protease (cleaves on the carboxyl side of aspartate and glutamate residues (glutamate only under certain conditions)). Enzymatic cleavage is discussed further as follows.

In preferred embodiments, the proteins in a sample are fragmented by one or more proteolytic enzymes (i.e., proteases, peptidases, proteinases, etc.). Proteolytic enzymes are hydrolases that catalyze the hydrolysis of peptide bonds (i.e., between the carboxylic acid group of one amino acid and the amino group of another) within protein molecules. Exemplary proteases suitable for use in the methods of the present invention are optionally selected from, e.g., aminopeptidases (EC 3.4.11), dipeptidases (EC 3.4.13), dipeptidyl-peptidases and tripeptidyl peptidases (EC 3.4.14), peptidyl-dipeptidases (EC 3.4.15), serine-type carboxypeptidases (EC 3.4.16), metallocarboxypeptidases (EC 3.4.17), cysteine-type carboxypeptidases (EC 3.4.18), omegapeptidases (EC 3.4.19), serine proteinases (EC 3.4.21), cysteine proteinases (EC 3.4.22), aspartic proteinases (EC 3.4.23), metallo proteinases (3.4.24), proteinases of unknown mechanism (EC 3.4.99), or the like. Additional description regarding these and other suitable enzymes is found on-line at, e.g., the ExPASy proteomics server (www.expasy.ch), the MEROPS database (www.merops.co.uk), or in the links thereto. Proteolytic enzymes are also described further in, e.g., Polgar, Mechanisms of Protease Action (1989) CRC Press, Boca Raton, Barrett et al. (Eds.), Handbook of Proteolytic Enzymes (1999) Academic Press, San Diego, Barrett et al. (Eds.), Methods in Enzymology: Proteolytic Enzymes: Aspartic and Metallo Peptidases (1995) Academic Press, San Diego, Springer and Stocker (Eds.), Proteolytic Enzymes: Tools and Targets (1999) Springer Verlag, New York, and Beynon and Bond, Proteolytic Enzymes: A Practical Approach (1989) IRL Press, Oxford.

Additional processing is optionally utilized if proteins in a sample include multiple polypeptide chains and/or include disulfide bonds. For example, if a protein includes multiple polypeptide chains held together by noncovalent bonds (e.g., electrostatic interactions or the like), denaturing agents, such as urea or guandine hydrochloride may be used to dissociate the polypeptide chains from one another prior to fragmentation. If a protein includes disulfide bonds, e.g., within a single polypeptide chain, and/or between distinct polypeptide chains, the disulfide bonds are optionally cleaved by reduction with thiols, such as dithiothreitol, β-mercaptoethanol, or the like. After reduction, cysteine residues from disulfide bonds are optionally alkylated with, e.g., iodoacetate to form S-carboxymethyl derivatives to prevent the disulfide bonds from reforming.

In certain embodiments of the invention, target proteins and/or peptide fragments resulting from fragmentation on are optionally modified to improve resolution upon detection. For instance, neuraminidase can be used to remove terminal sialic acid residues from glycoproteins to improve binding to an anionic adsorbent (e.g., cationic exchange ProteinChip® arrays) and to improve detection resolution. In another example, the target proteins and/or peptide fragments can be modified by the attachment of a tag of a particular molecular weight that specifically binds to these biomolecules to further distinguish them.

In other embodiments, the fragmentation of the first sample can be performed “on chip” in a SELDI environment by placing an aliquot of the sample on an adsorbent spot and adding the proteolytic agent to the material on the spot.

III. Profiling Peptide Fragments

The sample comprising the peptide fragments generated after fragmentation is referred to here as the “fragmented sample.” The fragmented sample is used to prepare aliquots, each subject to a different conditions for further analysis by gas phase ion spectrometry. A first aliquot of the sample is not subject to further fractionation and can be analyzed “as is.” Second and, optionally, third, fourth, etc., aliquots are subject to fractionation of the peptide fragments, generating sub-samples which contain fragments of the target protein, but which are less complex in their complement of peptides to be examined. Generally, the fractionation methods that, generate the second, third, fourth, etc. sub-samples are different, resulting in different populations of peptide fragments in each sub-fraction. The second, third, etc. aliquots are typically analyzed using, e.g., retentate chromatography. The advantage of further fractionation of the fragmented sample is that by collecting a sub-set of the peptide fragments into the sub-samples, the fractionation step reduces the complexity of the resulting sample. Reduced complexity results in an improved ability to detect and resolve fragments of the target protein that are not detectable in the fragmented sample due to a variety of conditions that suppress the signal of that peptide fragment. For example, a rare peptide fragment in the fragmented sample may become more predominant and detectable following further fractionation of a particular sample aliquot.

A variety of fractionation and analytic methods are useful and will be described below. However, in a preferred embodiment, the analysis of the peptide fragments of the first aliquot is performed by SELDI, and the fractionation and analysis of peptide fragments in the second, third, fourth, etc. aliquots is performed by retentate chromatography. A review of SELDI and retentate chromatography are now appropriate.

SELDI, or “surface-enhanced laser desorption/ionization,” is a method of gas phase ion spectrometry in which the surface of substrate which presents the analyte to the energy source plays all active role in the desorption and ionization process. The SELDI technology is described in, e.g., U.S. Pat. No. 5,719,060 (Hutchens and Yip). Retentate chromatography is a process for fractionating biomolecules on a solid phase adsorbent and analyzing the fractionated molecules by SELDI. Retentate chromatography is described in, e.g., International Publication WO 98/59360 (Hutchens and Yip).

A. SELDI and MALDI

SELDI differs from MALDI in the participation of the sample presenting surface in the desorption/ionization process. In MALDI, the sample presenting surface plays no role in this process—the analytes detected reflect those mixed with and trapped within the matrix material. In SELDI, the sample presenting surface comprises adsorbent molecules that exhibit some level of affinity for certain classes of analyte molecules. Thus, after application of energy absorbing molecules (e.g., “matrix”) to the surface and impingement by an energy source, the specific analyte molecules detected depend, in part, upon the interaction between the adsorbent and the analyte molecules. Thus, different populations of molecules are detected when performing SELDI and MALDI.

Three different versions of SELDI are described here: “Retentate Chromatography,” “No-wash SELDI” and “Concentration SELDI.”

1. Retentate Chromatography

Retentate chromatography generally proceeds as follows. A liquid sample comprising bioorganic analytes is applied to a sample presenting surface which comprises an adsorbent, e.g., a spot on the surface of a biochip. The adsorbent possesses various levels of affinity for classes of molecular analytes based on chemical characteristics. For example, a hydrophilic adsorbent has affinity for hydrophilic biomolecules. The sample is allowed to reach binding equilibrium with the adsorbent. In reaching binding equilibrium, the analytes bind to the adsorbent or remain in solution based on their level of attraction to the adsorbent.

The particular binding equilibrium struck by a class of molecules is, of course, mediated by the binding constant of that molecule for the adsorbent: The smaller the binding constant, the tighter the binding between the molecule and the adsorbent and the more likely the molecule is to be bound to the adsorbent than to be in solution. Molecules that are non-attracted or repelled by the adsorbent are likely to be free in solution, with few, if any, being bound to the adsorbent.

After allowing molecules to bind to the adsorbent, the liquid and unbound molecules are removed from the spot, e.g., by pipetting. What is left on the spot are molecules bound to the adsorbent and probably some unbound molecules not completely removed with the liquid. Thus, most of the unbound molecules are removed with the removal of the liquid.

Then, a wish solution is applied to the spot. Generally, the wash solution has a different elution characteristic than the liquid in which the sample was applied. For example, the wash solution may have a different pH or salt concentration than that of the original sample. In the wash step, the analytes may reach a new equilibrium between being bound and remaining in solution. For example, if the stringency of the wash is greater than the stringency of the liquid in which the sample was applied, weakly bound molecules may be released into solution. This wash solution is now removed from the spot, taking with it unbound molecules. This includes both biomolecular analytes as well as inorganic molecules such as salts. Thus, the wash can function as a desalting step, particularly if the wash solution has similar characteristics to the solution in which the sample was applied.

After the wash step, the population of analyte molecules on the surface is significantly different from that of the population in the original sample. In particular, compared with molecules in the original sample, the ratio of molecules remaining on the adsorbent is heavily skewed toward those with particular affinity for the adsorbent, and molecules that have little or no affinity for the adsorbent have been removed by washing.

At this point, the analytes remaining on the surface are usually allowed to dry, although this step is not necessary. The analytes now exist as a layer on the spot.

Energy absorbing molecules, sometimes called matrix, are applied to the probe surface to facilitate desorption/ionization. Usually, the energy absorbing molecules are applied to the spot and allowed to dry. However, in some embodiments, the energy absorbing molecules are applied to the surface of the probe before application of the sample. (One version of this embodiment is called “SEND.” See U.S. Pat. No. 6,124,137 (Hutchens and Yip).) The analytes can now be examined by gas phase ion spectrometry, preferably laser desorption/ionization mass spectrometry; the interaction between the matrix and the surface layer of analytes at the interface between the two enabling desorption and ionization of biomolecular analytes at this interface.

2. No-Wash SELDI

Another method, “No-wash SELDI,” includes the following steps: A liquid sample comprising bioorganic analytes is applied to a sample presenting surface which comprises an adsorbent, e.g., a spot on the surface of a biochip. The sample is allowed to reach equilibrium with the adsorbent. After allowing molecules to bind to the adsorbent, the liquid is removed from the spot, e.g., by pipetting or the like. The bound molecules (and probably some unbound molecules) remain on the substrate and most of the unbound molecules are removed with the liquid. In this method, no wash solution is applied to the spot. Because excess sample is removed after reaching equilibrium, and without a wash step, the population of molecules on the adsorbent spot differs from the population of molecules in the applied sample and from the population remaining on the spot in retentate chromatography. As in retentate chromatography, the population on the adsorbent spot is richer in molecules having affinity for the adsorbent, compared with the originally applied sample. However, the population also differs from that remaining in retentate chromatography because un-bound, non-specifically bound or weakly bound molecules, which are washed away in retentate chromatography, remain on the sample presenting surface. This includes both biomolecular and inorganic species, such as salts.

At this point, the analytes remaining on the surface are usually allowed to dry, although this step is not necessary. Then, an energy absorbing material (e.g., a cinnamic acid derivative, sinapinic acid and dihydroxybenzoic acid) is applied to the spot and allowed to dry. Then the analytes can be examined by gas phase ion spectrometry, preferably laser desorption/ionization mass spectrometry.

3. Concentration SELDI

In another method, referred to as “Concentration SELDI,” the steps proceed as follows. A liquid sample comprising bioorganic analytes is applied to a sample presenting surface which comprises an adsorbent, e.g., a spot on the surface of a biochip. The analytes in the sample are now concentrated on the adsorbent surface. Concentration proceeds by reducing the volume of the sample (e.g., by evaporation) so that the amount of analyte per unit volume increases. In contrast to No-wash SELDI or Retentate chromatography, sample liquid and unbound analytes are not removed together from the adsorbent surface. The analytes in the sample are preferably concentrated essentially to dryness. However, concentration can proceed at least 2-fold, at least 10-fold, at least 100-fold, or at least 1000 fold before application of energy absorbing molecules. Because the volume of the sample decreases steadily, the analytes never reach a stable binding equilibrium in solution. By concentrating the analytes on the adsorbent, all the analytes in the sample remain on the surface, regardless of their attraction to the adsorbent. (Certain volatile analytes may be lost in an evaporation process.) Thus, there is both specific binding (i.e., adsorbent mediated) and non-specific binding of analytes to the adsorbent surface. Then, an energy absorbing material is applied to the spot and allowed to dry. Then the analytes can,be examined by gas phase ion spectrometry, preferably laser desorption/ionization mass spectrometry.

In this case, while the population of analytes on the surface of the chip reflects the population of analytes in the applied sample, a fraction of the analytes remain bound to the chip surface even after the application of an energy absorbing material. Thus, the analyte fraction incorporated into the energy absorbing material represents the fraction of analytes which have low binding affinity for the adsorbent surface under the conditions present when the solution of energy absorbing material is deposited on the adsorbent surface. This contrasts with MALDI, in which the analyte sample is mixed directly with matrix material. The result is that signal strength from an analyte in each case is different, and signals from certain molecules, which are not detectable or distinguishable in MALDI can be detected in concentration SELDI. Thus, concentration SELDI can provide a more sensitive assay for the presence of bioorganic molecules in a sample than MALDI.

B. Contacting a Fragmented Sample with a Substrate for Gas Phase Ion Spectrometeric Analysis

1. Analysis of an Unfractionated “First Aliquot”

A “first aliquot,” that is, all aliquot of the fragmented sample that has not been subject to further fractionation, is examined by gas phase ion spectrometry, e.g., MALDI or SELDI.

In MALDI, the sample is usually mixed with an appropriate matrix, placed on the surface of a probe and examined by laser desorption/ionization. The technique of MALDI is well known in the art. See, e.g., U.S. Pat. No. 5,045,694 (Beavis et al.), U.S. Pat. No. 5,202,561 (Gleissmann et al.), and U.S. Pat. No. 6,111,251 (Hillenkamp). However, MALDI frequently does not provide results as good as analysis by SELDI.

In SELDI, the first aliquot is contacted with a solid phase-bound (e.g., substrate-bound) adsorbent. A substrate is typically a probe (e.g., a biochip) that is removably insertable into a gas phase ion spectrometer. In SELDI-based applications of the present invention, a probe generally includes a substrate with at least one surface feature having at least one adsorbent, bound to the substrate, that is capable of capturing, e.g., one or more peptide fragments from target proteins. A preferred adsorbent for this application is a normal phase or hydrophilic adsorbent, e.g., silicon oxide. Probes are described in greater detail below.

Alternatively, the substrate can be a solid phase, such as a polymeric, paramagnetic, latex, or glass bead or resin comprising, e.g., a functional group or adsorbent for binding peptide fragments. After capture of the analyte, the solid phase is placed on a probe that is removably insertable into a gas phase ion spectrometer.

An aliquot is contacted with a probe comprising an adsorbent, by any suitable manner, such as bathing, soaking, dipping, spraying, washing over, pipetting, etc. Generally, a volume of a sample aliquot containing from a few attomoles to 100 picomoles of peptide fragments in about 1 μl to 500 μl of a solvent is sufficient for binding to an adsorbent. The sample aliquot can contact the probe substrate comprising an adsorbent for a period of time sufficient to allow peptide fragments to bind to the adsorbent. Typically, the sample aliquot and a substrate comprising an adsorbent are contacted for a period of between about 30 seconds and about 12 hours, and preferably, between about 30 seconds and about 15 minutes. Furthermore, the sample aliquot is generally contacted to the probe substrate under ambient temperature and pressure conditions. For some sample aliquots, however, modified temperature (typically 4° C. through 37° C.) and pressure conditions can be desirable, which conditions ale determinable by those skilled in the art.

The sample is allowed to dry on the spot, or, after a suitable time, the excess sample is removed from the spot. Thereafter, peptide fragments in the first aliquot are desorbed and ionized from the probe and detected using gas phase ion spectrometry to provide a first set of peptide fragment mass data. The first set of peptide fragment mass data generally provides a profile of all or most peptide fragments present in the sample aliquot.

2. Analysis of the Fractionated “Second Aliquot”

Subsequent aliquots of a given fragmented protein sample are analyzed, according to the methods of the present invention, after fractionation of at least one aliquot of the fragmented sample. Fractionation of an aliquot increases the total information content about the peptide fragments in the fragmented sample. First, fractionation results in the detection of peptide fragments which were previously undetectable or not accurately detected in the fragmented sample by eliminating signals from more abundant peptide fragments that suppress the signal of less abundant peptide fragments. Second, the peptide fragments remaining in the sample after fractionation can be detected with better mass accuracy as a result of an increased signal:noise ratio. The use of information about peptide fragments from the fractionated sample as well as unfractionated, fragmented sample generally leads to a higher confidence level that a given target protein has been accurately identified by a database query based upon detected peptide fragments.

The fractionation steps that generate sub-samples from the second, third, etc. aliquots can be performed by any of the fractionation methods described above. For example, prior to spectrometrically profiling peptide fragment masses in a particular aliquot, biomolecules in the aliquot are separated into one or more sub-samples using, e.g., HPLC. In a preferred embodiment, the fractionation and analysis is performed by SELDI/retentate chromatography, which is now described in more detail.

In one embodiment, these fractionated aliquots are now analyzed by typical MALDI methods, such as those described above, in which the sample is applied to a probe surface that is not actively involved in the desorption/ionization of the analyte from the probe surface.

However, in a preferred embodiment, fractionating and analyzing the sample aliquot is performed by retentate chromatography. Retentate chromatography involves directly contacting an aliquot with an adsorbent bound to a surface of a probe in which the adsorbent captures one or more peptide fragments from the target protein. This embodiment also includes removing non-captured material from the probe, e.g., by one or more washes prior to gas phase ion spectrometeric analysis. Optionally, the aliquot is indirectly contacted with a probe surface after being contacted with a support-bound adsorbent that captures one or more peptide fragments derived from the target protein. Non-captured materials ale optionally removed (e.g., by one or more washes) before or after the support-bound adsorbent is contacted with the probe surface.

Washing to remove non-captured materials can be accomplished by, e.g., bathing, soaking, dipping, rinsing, spraying, or washing the substrate surface, or a support-bound adsorbent, following exposure to the sample aliquot with an eluant. A microfluidics process is preferably used when an eluant is introduced to small spots (e.g., surface features) of adsorbents on the probe. Typically, the eluant can be at a temperature of between 0° C. and 100° C., preferably between 4° C. and 37° C. Any suitable eluant (e.g., organic or aqueous) can be used to wash the substrate surface. For example, each of the one or more washes optionally includes an identical or a different elution condition relative to at least one preceding wash. Elution conditions typically differ according to, e.g., pH, buffering capacity, ionic strength, a water structure characteristic, detergent type, detergent strength, hydrophobicity, dielectric constant, concentration of at least one solute, or the like. Preferably, an aqueous solution is used. Exemplary aqueous solutions include a HEPES buffer, a Tris buffer, or a phosphate buffered saline, etc. To increase the wash stringency of the buffers, additives can be incorporated into the buffers. These include, but are not limited to, ionic interaction modifiers (both ionic strength and pH), water structure modifiers, hydrophobic interaction modifiers, chaotropic reagents, affinity interaction displacers. Specific examples of these additives can be found in, e.g., PCT publication WO98/59360 (Hutchens and Yip). The selection of a particular eluant or eluant additives is dependent on other experimental conditions (e.g., types of adsorbents used or peptide fragments to be detected), and can be determined by those of skill in the art.

Prior to desorption and ionization of biomolecules including peptide fragments from a probe surface according to ally of the methods described herein, an energy absorbing molecule (“EAM”) or a matrix material is typically applied to a given aliquot or sub-sample on the substrate surface, usually after allowing the sample to dry. The energy absorbing molecules can assist absorption of energy from an energy source from a gas phase ion spectrometer, and can assist desorption of peptide fragments from the probe surface. Exemplary energy absorbing molecules include cinnamic acid derivatives, sinapinic acid (“SPA”), cyano hydroxy cinnamic acid (“CHCA”) and dihydroxybenzoic acid. Other suitable energy absorbing molecules are known to those skilled in the art. See, e.g., U.S. Pat. No. 5,719,060 (Hutchens & Yip) for additional description of energy absorbing molecules.

The energy absorbing molecule and the peptide fragments in a given sample aliquot, or sub-sample of an aliquot (e.g., following further fractionation of the aliquot) can be contacted in any suitable manner. For example, an energy absorbing molecule is optionally mixed with a sample aliquot, or sub-sample of an, aliquot containing peptide fragments, and the mixture is placed on the substrate surface, as in traditional MALDI process. In another example, an energy absorbing molecule can be placed on the substrate surface prior to contacting the substrate surface with a sample aliquot, or sub-sample of an aliquot. In another example, a sample aliquot, or sub-sample of an aliquot, can be placed on the substrate surface prior to contacting the substrate surface with all energy absorbing molecule. Then, the peptide fragments can be desorbed, ionized and detected as described in detail below.

The analysis of the first and second aliquots preferably is performed in parallel, that is by dividing the fragmented sample into two aliquots and examining a first aliquot directly and a second aliquot after fractionation. However, in other embodiments of the invention, the analysis can be performed in series. For example, the first aliquot can be placed on a spot and allowed to equilibrated. Then the remaining liquid can be treated as the “second aliquot” by transferring it to an adsorbent spot for fractionation by retentate chromatography.

3. Probes

A probe (e.g., a biochip) is optionally formed in any suitable shape (e.g., a square, a rectangle, a circle, or the like) as long as it is adapted for use with a gas phase ion spectrometer (e.g., removably insertable into a gas phase ion spectrometer). For example, the probe call be in the form of a strip, a plate, or a dish with a series of wells at predetermined addressable locations or have other surfaces features. The probe is also optionally shaped for use with inlet systems and detectors of a gas phase ion spectrometer. For example, the probe can be adapted for mounting in a horizontally, vertically and/or rotationally translatable carriage that horizontally, vertically and/or rotationally moves the probe to a successive position without requiring repositioning of the probe by hand.

In certain embodiments, the probe substrate surface can be conditioned to bind analytes. For example, in one embodiment; the surface of the probe substrate call be conditioned (e.g., chemically or mechanically such as roughening) to place adsorbents on the surface. The adsorbent comprises functional groups for binding with an analyte. In some embodiments, the substrate material itself can also contribute to adsorbent properties and may be considered part of an “adsorbent.”

Adsorbents can be placed on the probe substrate in continuous or discontinuous patterns. If Continuous, one or more adsorbents can be placed on the substrate surface. If multiple types of adsorbents are used, the substrate surface can be coated such that one or more binding characteristics vary in a one- or two-dimensional gradient. If discontinuous, plural adsorbents cain be placed in predetermined addressable locations or surface features (e.g., addressable by a laser beam of a mass spectrometer) on the substrate surface. The surface features of probes or biochips include various embodiments. For example, a biochip optionally includes a plurality of surface features arranged in, e.g., a line, an orthogonal array, a circle, or an n-sided polygon, wherein n is three or greater. The plurality of surface features typically includes a logical or spatial array. Optionally, each of the plurality of surface features comprises identical or different adsorbents, or one or more combinations thereof. For example, at least two of the plurality of surface features optionally includes identical or different adsorbents, or one or more combinations thereof. Suitable adsorbents are described in greater detail below.

The probe substrate can be made of any suitable material. Probe substrates are preferably made of materials that are capable of supporting adsorbents. For example, the probe substrate material can include, but is not limited to, insulating materials (e.g., plastic, ceramic, glass, or the like), a magnetic material, semi-conducting materials (e.g., silicon wafers), or electrically conducting materials (e.g., metals, such as nickel, brass, steel, aluminum, gold, metalloids, alloys or electrically conductive polymers), polymers, organic polymers, conductive polymers, biopolymers, native biopolymers, metal coated with organic polymers, or any combinations thereof. The probe substrate material is also optionally solid or porous.

Probes are optionally produced using any suitable method depending on the selection of substrate materials and/or adsorbents. For example, the surface of a metal substrate can be coated with a material that allows derivatization of the metal surface. More specifically, a metal surface can be coated with silicon oxide, titanium oxide, or gold. Then, the surface can be derivatized with a bifunctional linker, one end of which can covalently bind with a functional group on the surface and the other end of which can be further derivatized with groups that function as an adsorbent. In another example, a porous silicon surface generated from crystalline silicon can be chemically modified to include adsorbents for binding analytes. In yet another example, adsorbents with a hydrogel backbone can be formed directly on the substrate surface by in situ polymerizing a monomer solution that includes, e.g., substituted acrylamide monomers, substituted acrylate monomers, or derivatives thereof comprising a selected functional group as an adsorbent. Probes suitable for use in the invention are described in, e.g., U.S. Pat. No. 5,617,060 (Hutchens and Yip) and WO 98/59360 (Hutchens and Yip).

4. Adsorbents

In some embodiments, the complexity of a sample aliquot can be further reduced using a substrate that comprises adsorbents capable of binding one or more peptide fragments. A plurality of adsorbents are optionally utilized in the methods of this invention. Different adsorbents can exhibit grossly different binding characteristics, somewhat different binding characteristics, or subtly different binding characteristics. For example, adsorbents need not be biospecific (e.g., biomolecular interaction adsorbents, such as antibodies that bind specific peptide fragments) as long as the adsorbents have binding characteristics suitable for binding a subset of peptide fragments with a particular characteristic from the sample. For example, adsorbents optionally include chromatographic adsorbents, such as a hydrophobic interaction adsorbent or group, a hydrophilic interaction adsorbent or group, a cationic adsorbent or group, an anionic adsorbent or group, a metal-chelating adsorbent or group (e.g., nickel, cobalt, etc.), lectin, heparin, or any combination thereof. In other embodiments, adsorbents include biomolecular interaction adsorbents, such as affinity adsorbents, polypeptides, enzymes, receptors, antibodies, or the like. For example, in certain embodiments, a biomolecular interaction adsorbent includes a monoclonal antibody that captures specific peptide fragments from a target protein.

Adsorbents which exhibit grossly different binding characteristics typically differ in their bases of attraction or mode of interaction. The basis of attraction is generally a function of chemical or biological molecular recognition. Bases for attraction between an adsorbent and an analyte, such as a peptide fragment include, e.g., (1) a salt-promoted interaction, e.g., hydrophobic interactions, thiophilic interactions, and immobilized dye interactions, (2) hydrogen bonding and/or van der Waals force interactions and charge transfer interactions, e.g., hydrophilic interactions, (3) electrostatic interactions, such as an ionic charge interaction, particularly positive or negative ionic charge interactions, (4) the ability of the analyte to form coordinate covalent bonds (i.e., coordination complex formation) with a metal ion on the adsorbent, or (5) combinations of two or more of the foregoing modes of interaction. That is, the adsorbent can exhibit two or more bases of attraction, and thus be known as a “mixed functionality” adsorbent.

a) Salt-Promoted Interaction Adsorbents

Adsorbents that ale useful for observing salt-promoted interactions include hydrophobic interaction adsorbents. Examples of hydrophobic interaction adsorbents include matrices having aliphatic hydrocarbons (e.g., C₁-C₁₈ aliphatic hydrocarbons) and matrices having aromatic hydrocarbon functional groups (e.g., phenyl groups). Another adsorbent useful for observing salt-promoted interactions includes thiophilic interaction adsorbents, such as T-Gel® which is one type of thiophilic adsorbent commercially available from Pierce, Rockford, Ill. A third adsorbent which involves salt-promoted ionic interactions and also hydrophobic interactions includes immobilized dye interaction adsorbents.

(i) Reverse Phase Adsorbent—Aliphatic Hydrocarbon

One useful reverse phase adsorbent is a hydrophobic adsorbent which is present on an H4 ProteinChip® array, available from Ciphergen Biosystems, Inc. (Fremont, Calif.). The hydrophobic H4 chip comprises aliphatic hydrocarbon chains immobilized on top of silicon oxide (SiO₂) as the adsorbent on the substrate surface.

b) Hydrophilic Interaction Adsorbents

Adsorbents which are useful for observing hydrogen bonding and/or van der Waals forces on the basis of hydrophilic interactions include surfaces comprising normal phase adsorbents such as silicon oxide (SiO₂). The normal phase or silicon-oxide surface acts as a functional group. In addition, adsorbents comprising surfaces modified with hydrophilic polymers Such as polyethylene glycol, dextran, agarose, or cellulose can also function as hydrophilic interaction adsorbents. Most proteins will bind hydrophilic interaction adsorbents because of a group or combination of amino acid residues (i.e., hydrophilic amino acid residues) that bind through hydrophilic interactions involving hydrogen bonding or van der Waals forces.

(i) Normal Phase Adsorbent—Silicon Oxide

One useful hydrophilic adsorbent is presented on a Normal Phase (NP) ProteinChip® array, available from Ciphergen Biosystems, Inc. (Fremont, Calif.). The normal phase chip comprises silicon oxide as the adsorbent on the substrate surface. Silicon oxide call be applied to the surface by any of a number of well known methods. These methods include, for example, vapor deposition, e.g., sputter coating. A preferred thickness for such a probe is about 9000 Angstroms.

c) Electrostatic Interaction Adsorbents

Adsorbents which are useful for observing electrostatic or ionic charge interactions include anionic adsorbents such as, for example, matrices of sulfate anions (i.e., SO₃⁻) and matrices of carboxylate anions (i.e., COO⁻) or phosphate anions (i.e., PO₄⁻). Matrices having sulfate anions have permanent negative charges. However, matrices having carboxylate anions have a negative charge only at a pH above their pKa. At a pH below the pKa, the matrices exhibit a substantially neutral charge. Suitable anionic adsorbents also include anionic adsorbents which are matrices having a combination of sulfate and carboxylate anions and phosphate anions.

Other adsorbents which are useful for observing electrostatic or ionic charge interactions include cationic adsorbents. Specific examples of cationic adsorbents include matrices of secondary, tertiary or quaternary amines. Quaternary amines are permanently positively charged. However, secondary and tertiary amines have charges that are pH dependent. At a pH below the pKa, secondary and tertiary amines are positively charged, and at a pH above their pKa, they are negatively charged. Suitable cationic adsorbents also include cationic adsorbents which are matrices having combinations of different secondary, tertiary, and quaternary amines.

In the case of ionic interaction adsorbents (both anionic and cationic) it is often desirable to use a mixed mode ionic adsorbent containing both anions and cations. Such adsorbents provide a continuous buffering capacity as a function of pH. Other adsorbents that are useful for observing electrostatic interactions include, e.g., dipole-dipole interaction adsorbents in which the interactions are electrostatic but no formal charge donor or acceptor is involved.

(i) Anionic Adsorbent

One useful adsorbent is an anionic adsorbent as presented on the SAX1 or SAX2 ProteinChip® array made by Ciphergen Biosystems, Inc. (Fremont, Calif.). The SAX1 protein chips are fabricated from SiO₂ coated aluminum substrates. In the process, a suspension of quaternary ammonium polystryenemicrospheres in distilled water is deposited onto the surface of the chip (1 mL/spot, two times). After air drying (room temperature, 5 minutes), the chip is rinsed with deionized water and air dried again (room temperature, 5 minutes).

(ii) Cationic Adsorbent

Another useful adsorbent is an cationic adsorbent as presented on the SCX1 or SCX2 ProteinChip® array made by Ciphergen Biosystems, Inc. (Fremont, Calif.). The SCX1 protein chips are fabricated from SiO₂ coated aluminum substrates. In the process, a suspension of sulfonate polystyrene microspheres in distilled water is deposited onto the surface of the chip (1 mL/spot, two times). After air drying (room temperature, 5 minutes), the chip is rinsed with deionized water and air dried again (room temperature, 5 minutes).

d) Coordinate Covalent Interaction Adsorbents

Adsorbents which are useful for observing the ability to form coordinate covalent bonds with metal ions include matrices bearing, for example, divalent and trivalent metal ions. Matrices of immobilized metal ion chelators provide immobilized synthetic organic molecules that have one or more electron donor groups which form the basis of coordinate covalent interactions with transition metal ions. The primary electron donor groups functioning as immobilized metal ion chelators include oxygen, nitrogen, and sulfur. The metal ions are bound to the immobilized metal ion chelators resulting in a metal ion complex having some number of remaining sites for interaction with electron donor groups on the analyte. Suitable metal ions include in general transition metal ions such as copper, nickel, cobalt, zinc, iron, and other metal ions Such is aluminum and calcium.

(i) Nickel Chelate Adsorbents

Another useful adsorbent is a metal chelate adsorbent as presented on the IMAC3 (Immobilized Metal Affinity Capture, nitrilotriacetic acid on surface) ProteinChip® array, also available from Ciphergen Biosystems, Inc. (Fremont, Calif.). The chips are produced as follows: 5-Methacylamido-2-(N,biscarboxymethaylamino)pentanoic acid (7.5 wt %), Acryloyltri(hydroxymethyl)methylamine (7.5 wt %), and N,N′-inethylenebisacrylamide (0.4 wt %) are photo-polymerized using (−)riboflavin (0.02 wt %) as a photo-initiator. The monomer solution is deposited onto a rough etched, glass coated substrate (0.4 mL, twice) and irradiated for 5 minutes with a near UV exposure system (Hg short arc lamp, 20 mW/cm² at 365 nm). The surface is washed with a solution of sodium chloride (1 M) and then washed twice with deionized water.

The IMAC3 with Ni(II) is activated as follows. The surface is treated with a solution of NiSO₄ (50 mM, 10 mL/spot) and mixed on a high frequency mixer for 10 minutes. After removing the NiSO₄ Solution, the treatment process is repeated. Finally, the surface is washed with a stream of deionized water (15 sec/chip).

e) Enzyme-Active Site Interaction Adsorbents

Adsorbents which are useful for observing enzyme-active site binding interactions include proteases (Such is trypsin), phosphatases, kinases, and nucleases. The interaction is a sequence-specific interaction of the enzyme binding site on the analyte (typically a biopolymer) with the catalytic binding site on the enzyme.

i) Reversible Covalent Interaction Adsorbents

Adsorbents which ale useful for observing reversible covalent interactions include disulfide exchange interaction adsorbents. Disulfide exchange interaction adsorbents include adsorbents comprising immobilized sulfhydryl groups, e.g., mercaptoethanol or immobilized dithiothreitol. The interaction is based upon the formation of covalent disulfide bonds between the adsorbent and solvent exposed cysteine residues on the analyte. Such adsorbents bind proteins or peptides having cysteine residues and nucleic acids including bases modified to contain reduced sulfur compounds.

g) Glycoprotein Interaction Adsorbents

Adsorbents which are useful for observing glycoprotein interactions include glycoprotein interaction adsorbents such as adsorbents having immobilize lectins (i.e., proteins bearing oligosaccharides) therein, an example of which is Conconavalin A, which is commercially available from, e.g., Sigma Chemical Company (St. Louis, Mo.). Such adsorbents function on the basis of the interaction involving molecular recognition of carbohydrate moieties on macromolecules.

h) Biospecific Interaction Adsorbents

Adsorbents which are useful for observing biospecific interactions are generically termed “biospecific affinity adsorbents.” Adsorption is considered biospecific if it is selective and the affinity (equilibrium dissociation constant, K_{d}) is at least 10⁻³ M to (e.g., 10⁻⁵ M, 10⁻⁷ M, 10⁻⁹ M, or the like). Examples of biospecific affinity adsorbents include any adsorbent which specifically interacts with and binds a particular biomolecule. Biospecific affinity adsorbents include for example, immobilized antibodies which bind to antigens, e.g., specific peptide fragments, immobilized receptors, or the like.

IV. Gas Phase Ion Spectrometry

In certain embodiments, peptide fragments present in a sample aliquot are detected using gas phase ion spectrometry, and more preferably, using mass spectrometry. In one embodiment, matrix-assisted laser desorption/ionization (“MALDI”) mass spectrometry is used, e.g., to profile peptide fragment masses in a first aliquot of the sample. In MALDI, the sample is typically quasi-purified (e.g., prior to protein fragmentation) to obtain a fraction that essentially consists of peptide fragments from a target protein using, e.g., protein separation methods such as two-dimensional gel electrophoresis, HPLC, or the like. Biomolecule fractionation techniques are described in greater detail above. Additional details relating to MALDI are included in, e.g., Skoog et al., Principles of Instrumental Analysis, 5^{th} Ed., Harcourt Brace & Co., Philadelphia (1998) and Siuzdak, Mass Spectrometry for Biotechnology, supra. Systems that include gas phase ion spectrometers are described further below.

In preferred embodiments, surface-enhanced laser desorption/ionization mass spectrometry is optionally used to desorb and ionize peptide fragments from probe surfaces. Surface enhanced laser desorption/ionization uses a substrate comprising adsorbents to capture peptide fragments, which are then optionally directly desorbed and ionized from the substrate surface during mass spectrometry. Since the substrate surface in surface enhanced laser desorption/ionization captures peptide fragments, a sample need not be quasi-purified as in MALDI. However, depending on the complexity of a sample and the type of adsorbents used, it is typically desirable to prepare a sample aliquot with reduced complexity by, e.g., removing non-captured materials prior to surface enhanced laser desorption/ionization analysis.

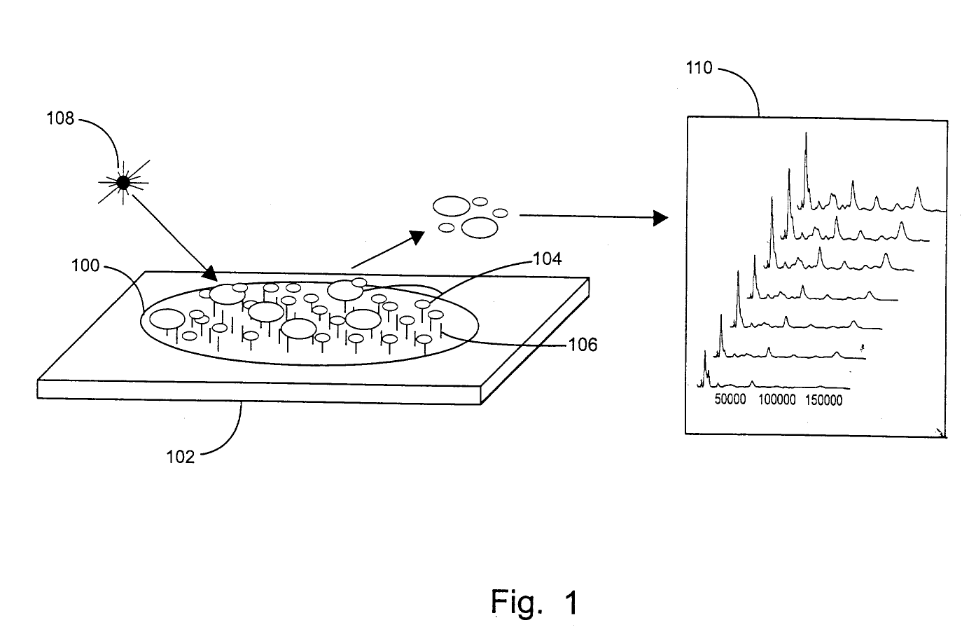

To illustrate, FIG. 1 schematically shows a surface enhanced laser desorption/ionization assay of all unfractionated first aliquot of a fragmented sample that includes chromatographic adsorbent 106 on biochip 102. Chromatographic adsorbents such as hydrophobic and hydrophilic interaction adsorbents are described further above. As additionally described above, peptide fragments 104 in the first aliquot are not washed after being placed on chromatographic adsorbent 106 which is bound to surface feature 100. Incident photon energy from laser 108 causes the desorption and ionization of peptide fragments 104, which are then detected in a mass spectrometer to produce mass spectra 110.

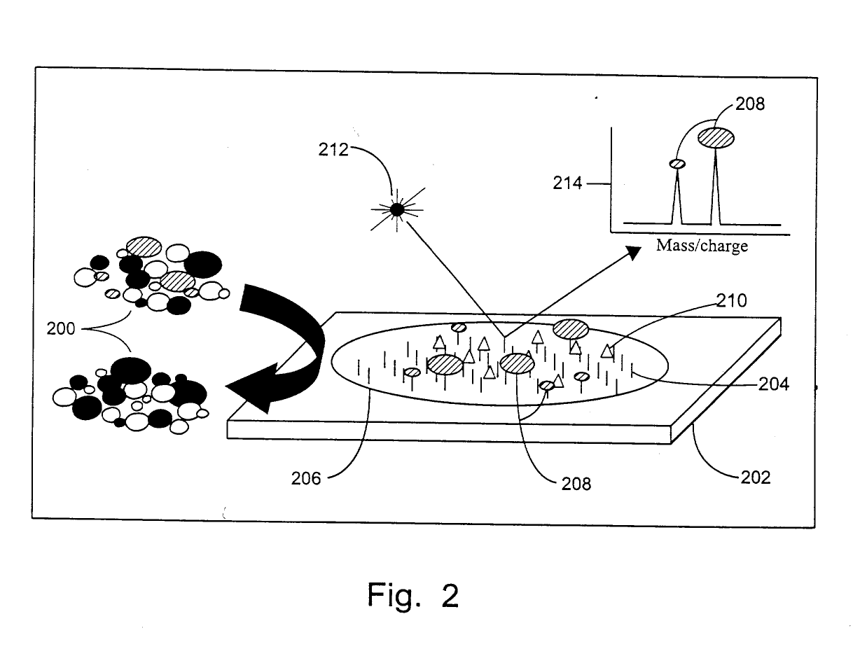

FIG. 2 schematically illustrates a surface enhanced laser desorption/ionization assay of a second or subsequent aliquot of a fragmented sample. As depicted, fragmented protein sample aliquot 200 is applied to biochip 202 which includes chromatographic adsorbent 204 bound to surface feature 206. Components of sample aliquot 200 that are not bound to chromatographic adsorbent 204 are washed away (e.g., eluted or the like) from biochip 202 prior to mass analysis, as described above. Following capture and washing of peptide fragments 208 in sample aliquot 200, energy absorbing molecules 210 (not shown in FIG. 1) are added to biochip 202 to absorb energy from ionization source 212 (i.e., a laser) to aid desorption of peptide fragments 208 from the surface of biochip 202. Mass spectrum 214 is produced by mass spectral analysis of desorbed/ionized peptide fragments 208.

Optionally, any suitable gas phase ion spectrometer is used as long as it allows peptide fragments on the substrate to be resolved and detected. For example, in certain embodiments the gas phase ion spectrometer is a mass spectrometer. In a typical mass spectrometer, a probe comprising peptide fragments on its surface is introduced into an inlet system of the mass spectrometer. The peptide fragments are then desorbed by a desorption source such as a laser, fast atom bombardment, high energy plasma, electrospray ionization, thermospray ionization, liquid secondary ion MS, field desorption, etc. The generated desorbed, volatilized species consist of preformed ions or neutrals which are ionized as a direct consequence of the desorption event. Generated ions ale collected by an ion optic assembly, and then a mass analyzer disperses and analyzes the passing ions. The ions exiting the mass analyzer are detected by a detector. The detector then translates information of the detected ions into mass-to-charge ratios. Detection of the presence of peptide fragments or other substances will typically involve detection of signal intensity. This, in turn, can reflect the quantity and character of peptide fragments bound to the substrate. Any of the components of a mass spectrometer (e.g., a desorption source, a mass analyzer, a detector, etc.) can be combined with other suitable components described herein or others known in the all in embodiments of the invention.

In preferred aspects, a laser desorption time-of-flight mass spectrometer is used in embodiments of the invention. In laser desorption mass spectrometry, a substrate or a probe comprising peptide fragments and/or other materials is introduced into an inlet system. The materials are desorbed and ionized into the gas phase by incident laser energy from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of peptide fragments of specific mass-to-charge ratios.

In another embodiment, an ion mobility spectrometer is optionally used to detect peptide fragments. The principle of ion mobility spectrometry is based on different ion mobilities. Specifically, ions of a sample produced by ionization move at different rates, clue to their difference in, e.g., mass, charge, or shape, through a tube under the influence of an electric field. The ions (typically in the form of a current) are registered at the detector which can then be used to identify a peptide fragment or other substance in a sample. One advantage of ion mobility spectrometry is that it can operate at atmospheric pressure.

In yet another embodiment, a total ion current measuring device is optionally used to detect and characterize peptide fragments. This device is optionally used when the substrate has only a single type of marker. When a single type of marker is on the substrate, the total Current generated from the ionized marker reflects the quantity and other characteristics of the marker. The total ion current produced by the marker can then be compared to a control (e.g., a total ion current of a known compound). The quantity or other characteristics of the marker can then be determined.

In still another embodiment, quadruple time-of-flight (Q-TOF) mass spectrometers, which are capable of tandem mass spectrometry, are optionally utilized to perform the methods described herein. These mass analyzer systems are readily coupled to laser desorption/ionization sources and are routinely used for protein and peptide analyses. Many Q-TOF mass spectrometers include mass ranges in excess of m/z 10000 and resolving powers of about 10000 full-width half maximum.

V. Data Analysis and Target Protein Identification

The data on peptide fragments detected by both the unfractionated “fragmented aliquot” and the fragmented “second aliquot,” “third aliquot,” etc. are now combined and analyzed to determine identity candidates for the target protein.

Data generated by desorption and detection of peptide fragments is optionally analyzed using any suitable method. In one embodiment, data is analyzed with the use of a logic device, such as a programmable digital computer that is included, e.g., as part of a system. Systems are described further below. The computer generally includes a computer readable medium that stores logic instructions of the system software. Certain logic instructions are typically devoted to memory that includes the location of each feature on a probe, the identity of the adsorbent at that feature, the elution conditions used to wash the adsorbent, or the like. The computer also typically includes logic instructions that receives as input, data on the strength of the signal at various molecular masses received from a particular addressable location or surface feature on the probe, and instructions for entering data into a database. This data generally indicates the number and masses of peptide fragments detected, including the strength of the signal generated by each fragment.

In preferred embodiments, the multiple sets of peptide fragment mass data (e.g., first set, second set, etc.) are in a computer-readable form suitable for use in database queries. For example, a database query generally includes operating the programmable computer or other logic device and executing an algorithm that determines closeness-of-fit between the computer-readable data and database entries. The database entries typically correspond to masses of identified proteins, or of peptide fragments from identified proteins, to produce at least one identity candidate for the target protein based upon one or more detected peptide fragment masses in the multiple sets of peptide fragment mass data. In preferred embodiments, the database query identifies the target protein. In some embodiments, the algorithm includes an artificial intelligence algorithm or a heuristic learning algorithm. For example, the artificial intelligence algorithm optionally includes one or more of, e.g., a fuzzy logic instruction set, a cluster analysis instruction set, a neural network, a genetic algorithm, or the like.

Essentially any protein database is optionally queried with peptide fragment mass data obtained using the methods and systems of the present invention. Many suitable databases are available and generally known in the art. For example, access to numerous protein databases and software for interfacing with these databases are available through the Expert Protein Analysis System (ExPASy) proteomics server of the Swiss Institute of Bioinformatics (www.expasy.ch). One of these databases is the SWISS-PROT database (www.ebi.ac.uk/swissprot/), which includes non-redundant sequence entries, high-quality annotation, and cross-references to many other databases. See, e.g., Junker et al. (2000) “The role SWISS-PROT and TrEMBL play in the genome research environment,” J. Biotechnol. 78(3):221-234 and Kriventseva et al. (2001) “CluSTr: a database of clusters of SWISS-PROT+TrEMBL proteins,” Nucleic Acids Res. 29(1):33-36. Additional description of protein databases and related subject matter is provided in, e.g., Rashidi and Buechler, Bioinformatics Basics: Applications in Biological Science and Medicine, CRC Press, Boca Raton (2000) and Pevzner, Computational Molecular Biology: An Algorithmic Approach, The MIT Press, Cambridge, Mass. (2000).

Various software packages are currently available for querying databases, improving the speed of the miss spectrometeric protein identification process, and otherwise integrating mass spectrometry into bioinformatics. For example, Mascot is a search engine that uses mass spectrometry data to identify proteins from primary sequence databases. See, e.g., Perkins et al. (1999) “Probability-based protein identification by searching sequence databases using mass spectrometry data,” Electrophoresis 20(18):3551-3567. Another exemplary software package that is useful for performing the methods of the present invention includes ProFound, which performs rapid database searching combined with Bayesian statistics for protein identification. Profound is described further in, e.g., Zhang and Chait (2000) “ProFound-An expert system for protein identification using mass spectrometeric peptide mapping information,” Anal. Chem. 72:2482-8249, Zhang and Chait (1998) “ProFound-An expert system for protein identification,” Proceedings of the 46th ASMS Conference on Mass Spectrometry and Allied Topics, Orlando, Fla., p.969, and Zhang and Chait (1995) “Protein identification by database searching: a Bayesian algorithm,” Proceedings of the 43rd ASMS Conference on Mass Spectrometry and Allied Topics, Atlanta, Ga., p. 643. Additional details regarding protein identification software packages suitable for performing the methods described herein are provided in, e.g., Jaffe and Pant (1998) “Characterization of serine and threonine phosphorylation sites in β-elimination/ethanediol addition-modified proteins by electrospray tandem mass spectrometry and database searching,” Biochemistry 37:16211-16224, Demirev et al. (1999) “Microorganism identification by mass spectrometry and protein database searching,” Anal. Chem. 71:2732-2738, Clauser et al. (1999) “Role of accurate mass measurement (−/− 10 ppm) in protein identification strategies employing MS or MS/MS and database searching,” Anal. Chem. 71:2871 -2882, and Green et al. (1999) “Mass accuracy and sequence requirements for protein database searching,” Anal. Biochem. 275:39-46.

Data analysis also generally includes the steps of determining signal strength (e.g., height of peaks) of an analyte detected and removing “outliers” (data deviating from a predetermined statistical distribution). The observed peaks can be normalized, a process whereby the height of each peak relative to some reference is calculated. For example, a reference can be background noise generated by an instrument and chemicals (e.g., energy absorbing molecules) which is set as zero in the scale. Then the signal strength detected for each marker or other biomolecules can be displayed in the form of relative intensities in the scale desired (e.g., 100). Alternatively, a standard (e.g., bovine serum albumin) may be admitted with the sample so that a peak from the standard can be used as a reference to calculate relative intensities of the signals observed for each peptide fragment or other biomolecules detected.

The computer can transform the resulting data into various formats for displaying. In one format, referred to as “spectrum view or retentate map,” a standard spectral view can be displayed, wherein the view depicts the quantity of peptide fragments or other biomolecules reaching the detector at each particular molecular weight. In another format, referred to as “peak map,” only the peak height and mass information are retained from the spectrum view, yielding a cleaner image and enabling analytes with nearly identical molecular weights to be more easily seen. In yet another format, refereed to as “gel view,” each mass from the peak view can be converted into a grayscale image based on the height of each peak, resulting in an appearance similar to bands on electrophoretic gels. In yet another format, referred to as “3-D overlays,” several spectra can be overlaid to study subtle changes in relative peak heights. In yet another format, referred to as “difference map view,” two or more spectra can be compared, conveniently highlighting unique analytes and analytes which are up- or down-regulated between samples. Peptide fragment profiles (spectra) from any two samples may be compared visually. In yet another format, a Spotfire Scatter Plot can be used in which peptide fragments that are detected are plotted as a dot in a plot, wherein one axis of the plot represents the apparent molecular weight of the fragments detected and another axis represents the signal intensity of fragments detected. For each sample, peptide fragments that are detected and the amount of fragments present in the sample call be saved in a computer readable medium. This data is then optionally compared to a control (e.g., a profile or quantity of peptide fragments detected in a control).

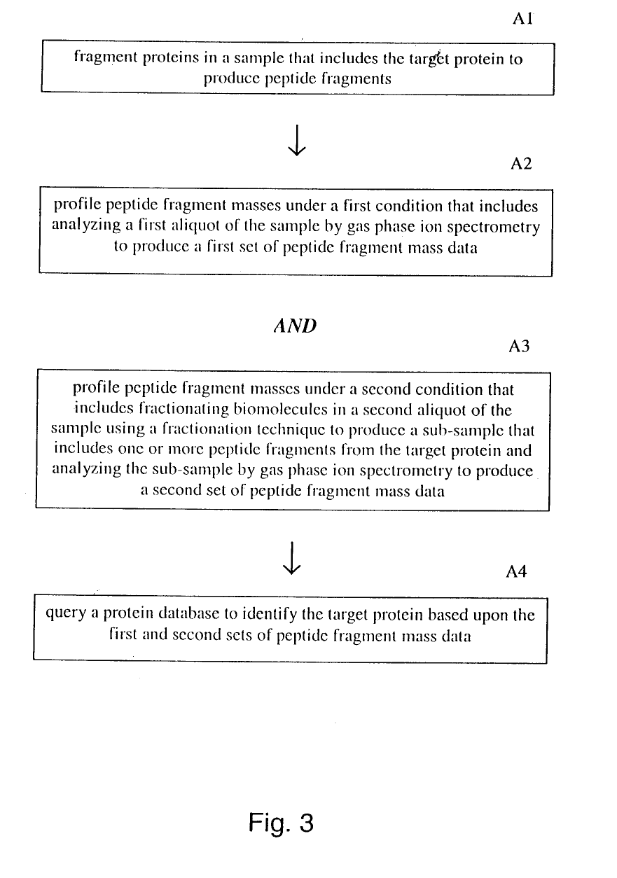

FIG. 3 is a flow chart that further schematically shows steps involved in methods of the invention for identifying a target protein based on two sets of peptide fragment mass data. Optionally, more than two sets of peptide fragment mass data are used (see, e.g., Example illustrating the identification of transferrin, below). As shown, the method includes A1, fragmenting proteins in a sample that includes the target protein to produce peptide fragments. Following A1, the method includes A2, profiling peptide fragment masses under a first condition that includes analyzing a first aliquot of the sample by gas phase ion spectrometry to produce a first set of peptide fragment mass data. The method also includes A3, profiling peptide fragment masses under a second condition that includes fractionating biomolecules in a second aliquot of the sample using a fractionation technique to produce a sub-sample that includes one or more peptide fragments from the target protein and analyzing the sub-sample by gas phase ion spectrometry to produce a second set of peptide fragment mass data. Finally, the method includes A4, querying a protein database to identify the target protein based upon the first and second sets of peptide fragment mass data. As with all of the methods described herein, one or more of these steps are typically effected under the direction of system software, which is discussed further below.

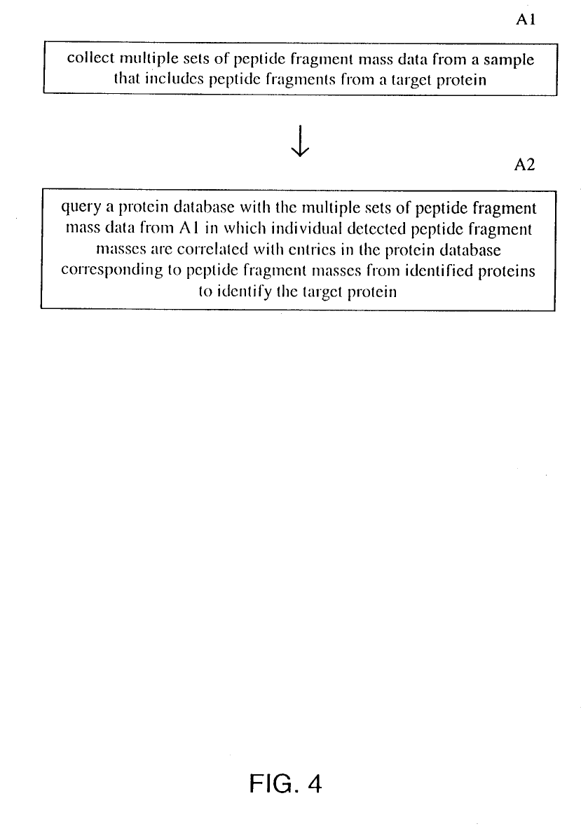

FIG. 4 is a flow chart that further schematically illustrates steps involved in one embodiment of a protein database query that involves multiple sets of peptide fragment mass data to identify a target protein. As shown, A1 includes collecting multiple sets of peptide fragment mass data from a sample that includes peptide fragments from a target protein. Thereafter, A2 involves querying a protein database with the multiple sets of peptide fragment mass data from A1 in which individual detected peptide fragment masses are correlated with entries in the protein database corresponding to peptide fragment masses from identified proteins to identify the target protein

The improved methods of the invention provide multiple sets of peptide fragment mass data to identify target proteins based upon the detected fragmentation patterns. If site-specific proteases, such as trypsin are used to fragment proteins in a sample, detected fragmentation patterns are predictable. Non-tandem mass spectrometry techniques are typically suitable to provide mass spectra corresponding to these predictable fragmentation patterns. If proteins are fragmented randomly, such as by a non-specific protease, by physical shearing, by certain chemical agents, or the like, a tandem mass spectrometry method (e.g., Q-TOF-MS) is generally used to provide sequence information about one or more of the peptide fragments included in the database query. In either case, that is, whether proteins are fragmented specifically or non-specifically, the increased number of peptide fragments and their mass accuracy detected according to the methods described herein increases the probability of finding all accurate match in the queried database.

VI. Protein Identification Systems

The present invention also provides a system capable of identifying target proteins in a sample based upon multiple sets of peptide fragment data according to the methods described herein. The system includes one or more adsorbents (e.g., adsorbents bound to a probe surface, support-bound adsorbents, or the like) capable of capturing peptide fragments derived from a target protein in the sample under at least two different conditions and a gas phase ion spectrometer (e.g., a mass spectrometer, such as a laser desorption/ionization mass spectrometer) able to profile masses of captured peptide fragments under the different conditions to provide multiple sets of peptide fragment mass data. That is, each data set corresponds to masses of peptide fragments detected under a different condition as described above. The system also includes a processor (e.g., in a computer or other logic device) operably connected to the gas phase ion spectrometer. The processor is optionally internal or external to the gas phase ion spectrometer. Optionally, the system includes multiple processors. System software typically includes logic instructions capable of determining closeness-of-fit between one or more detected peptide fragment masses in the sets of peptide fragment mass data and database entries. As described above, the database entries correspond to masses of identified proteins or peptide fragments from the identified proteins. Database queries typically produce at least one identity candidate for the target protein based upon the sets of peptide fragment mass data.

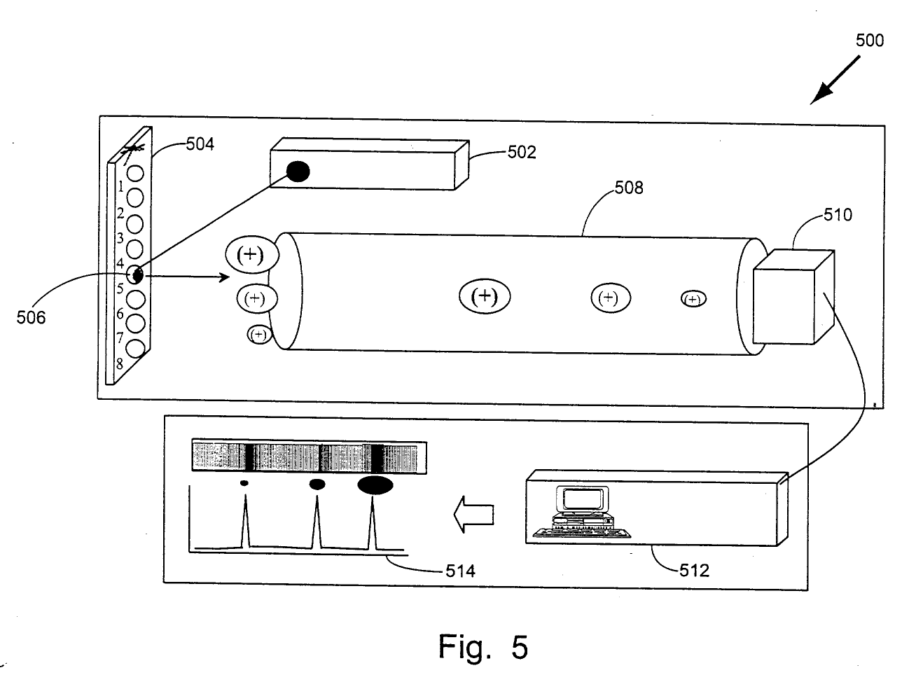

FIG. 5 schematically illustrates surface enhanced laser desolation/ionization time-of-flight mass spectrometry system 500. As shown, photon energy produced by laser source 502 impacts biochip 504 at surface feature 506, which includes a selected adsorbent with captured peptide fragments. The photon energy causes captured peptide fragments at surface feature 506 to desorb and ionize. The desorbed ions are then accelerated through flight tube/mass analyzer 508. Ions are separated according to mass/charge ratios, which as depicted is simply the mass of the ionic species, because each ion is singly charged. As further illustrated, smaller ions travel faster than larger ions, thereby resolving the species according to mass. Ions produce a detectable signal at detector 510 which signal is processed by information appliance or digital device 512 to generate mass spectrum 514.

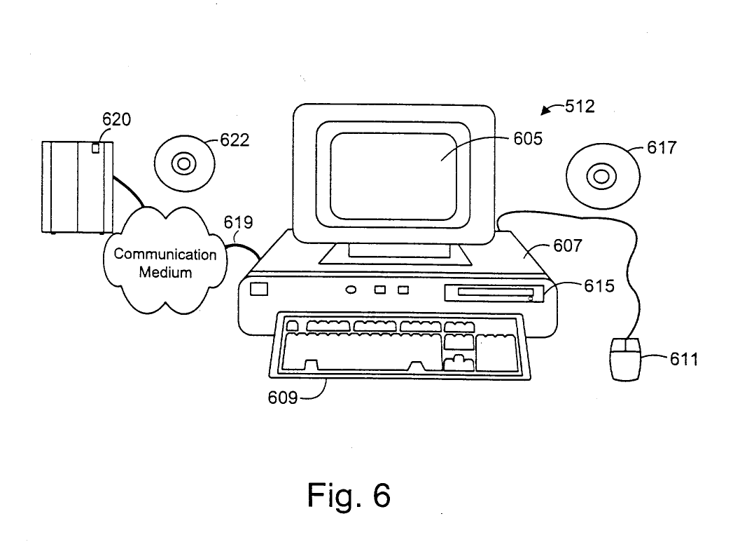

FIG. 6 is a schematic showing additional representative details of information appliance 512 from FIG. 5 in which various aspects of the present invention may be embodied. As will be understood by practitioners in the art from the teachings provided herein, the invention is optionally implemented in hardware and/or software. In some embodiments, different aspects of the invention are implemented in either client-side logic or server-side logic. As will be understood in the art, the invention or components thereof may be embodied in a media program component (e.g., a fixed media component) containing logic instructions and/or data that, when loaded into an appropriately configured computing device, cause that device to perform according to the invention. As will also be understood in the art, a fixed media containing logic instructions may be delivered to a viewer on a fixed media for physically loading into a viewer's computer or a fixed media containing logic instructions may reside on a remote server that a viewer accesses through a communication medium in order to download a program component.

FIG. 6 shows information appliance or digital device 512 that may be understood as a logical apparatus that can read instructions from media 617 and/or network port 619, which can optionally be connected to server 620 having fixed media 622. Apparatus 512 can thereafter use those instructions to direct server or client logic, as understood in the art, to embody aspects of the invention. One type of logical apparatus that may embody the invention is a computer system as illustrated in 512, containing CPU 607, optional input devices 609 and 611, disk drives 615 and optional monitor 605. Fixed media 617, or fixed media 622 over port 619, may be used to program such a system and may represent a disk-type optical or magnetic media, magnetic tape, solid state dynamic or static memory, or the like. In specific embodiments, the invention may be embodied in whole or in part as software recorded on this fixed media. Communication port 619 may also be used to initially receive instructions that are used to program such a system and may represent any type of communication connection. Optionally, the invention is embodied in whole or in part within the circuitry of al application specific integrated circuit (ACIS) or a programmable logic device (PLD). In such a case, the invention may be embodied in a computer understandable descriptor language, which may be used to create an ASIC, or PLD.

VII. Kits

In another aspect, the invention provides kits for identifying target proteins in samples according to the methods of the invention. In one embodiment, a kit includes (a) at least one adsorbent that captures peptide fragments, (b) a set of instructions for capturing peptide fragments from a sample by exposing the sample to the adsorbent and for profiling masses of the captured peptide fragments by gas phase ion spectrometry, and (c) at least one container for packaging the adsorbent and the set of instructions. Optionally, the kit also includes at least one eluant for washing the adsorbent to remove material other than the captured peptide fragments. The adsorbent typically includes a solid phase adsorbent. In one embodiment, the solid phase adsorbent is provided as a biochip that includes a substrate with at least one surface feature having the solid phase adsorbent bound to the substrate. The substrate is generally a probe adapted for use with a gas phase ion spectrometer. The kit optionally includes the probe.

In certain embodiments, the probe includes a substrate with a plurality of surface features. For example, each of the plurality of surface features optionally includes one or more adsorbent bound to the substrate. Optionally, one or more of the surface features lacks an adsorbent bound thereto. The plurality of surface features is generally arranged in a line, an orthogonal array, a circle, or an n-sided polygon, wherein n is three or greater. Optionally, the plurality of surface features includes a logical or spatial array. In other embodiments, the solid phase adsorbent includes a bead or resin derivatized with the adsorbent. For example, the bead or resin derivatized with the at least one adsorbent is typically suitable for being placed on a probe adapted for use with a gas phase ion spectrometer. As an additional option, the kit also includes at least one reference or control. In yet another embodiment, the kit may further comprise a pre-fractionation spin column (e.g., K-30 size exclusion column).

The kits of the present invention include various types of adsorbents. For example, in some embodiments, the adsorbent includes a chromatographic adsorbent, such as an anionic adsorbent, a cationic adsorbent, a hydrophobic interaction adsorbent, a hydrophilic interaction adsorbent (e.g., silicon oxide, etc.), a metal-chelating adsorbent (e.g., nickel, cobalt, etc.) or the like. In other embodiments, the adsorbent includes a biomolecular interaction adsorbent, such as an affinity adsorbent, a polypeptide, an enzyme, a prostatic marker substrate, a receptor, an antibody, or the like. In preferred embodiments, the biomolecular interaction adsorbent includes a monoclonal antibody that captures specific peptide fragments. In still other embodiments, the kit further includes multiple adsorbents. As an additional option, the kit also includes (1) an eluant in which peptide fragments are retained on the adsorbent when washed with the eluant, or (2) instructions to wash the adsorbent with the eluant after contacting the adsorbent with a sample.

Optionally, the kit further comprises instructions for suitable operational parameters in the form of a label or a separate insert. For example, the kit may have standard instructions informing a consumer how to wash the probe after, e.g., a sample aliquot is contacted on the probe. In another example, the kit may have instructions for pre-fractionating a sample to reduce complexity of proteins or other biomolecules in the sample. In yet another example, the kit optionally includes chemicals (e.g., CNBr, O-lodosobenxoate, etc.) and/or enzymes (e.g., trypsin or other proteases), and instructions for their use in fragmenting proteins in a sample prior to spectrometeric analysis.

VIII. EXAMPLE

The following non-limiting example is offered only by way of illustration.

A. Comparison of MALDI and SELDI Methods in Peptide Mapping

1. Overview

The accuracy of protein identification generally improves as the number of peptide fragments detected from, e.g., a protease digestion of a target protein is increased. Protein identification confidence levels also typically increase with improved accuracy of detected peptide fragment masses. One way to improve the accuracy of detected masses is to increase the signal-to-noise ratio of the analytical measurement. The present example illustrates that the methods of the present invention for peptide mapping achieve both increased numbers of detected peptide fragments and improved accuracy of detected individual fragment masses relative to those obtained by techniques, such as MALDI.

The analyses described in this example were performed using a ProteinChip® system (series PBS II), available from Ciphergen Biosystems, Inc. (Fremont, Calif.), which includes a ProteinChip® reader integrated with ProteinChip® software and a personal computer for analyzing detected peptide fragment masses. The ProteinChip® system is capable of detecting biomolecules ranging from less than about 1000 Da up to about 300 kilodaltons or more and calculates the masses based on time-of-flight. The ProteinChip® reader is a laser desolation/ionization time-of-flight mass spectrometer. The ion optics of the Reader are derived from a four-stage, time-lag-focusing ion lens assembly that provides precise, accurate molecular weight determination with excellent mass resolving power. The laser optics have been modified to maximize ion extraction efficiency over the greatest possible sample area, thus increasing analytical sensitivity and reproducibility.

Peptide fragments were generated by typtic digests of a purified and heat-denatured transferrin (bovine) and were used for both the MALDI and SELDI analyses. For the MALDI analysis, a gold allay was used to analyze a mixture of 1 μl of the peptide fragments and 1 μl of 20% saturated cyano hydroxy cinnamic acid (CHCA) in 50% acetonitrile and 0.1% trifluloroacetic acid (TFA). For the SELDI analysis performed according to the methods of the invention, a hydrophobic (H4) ProteinChip® array was used. Surface features were initially treated with 50% acetonitrile for 5 minutes prior to being contacted by peptide fragment sample aliquots. At a first surface feature (spot #1) of the array, 1 μl of the peptide fragments was applied and allowed to dry. Then, 1 μl of 20% saturated CHCA in 50% acetonitrile and 0.1% TFA was applied and mixed. At a second surface feature (spot #2) of the array, 1 μl of the peptide fragments was applied and allowed to dry. Spot #2 was washed three times with 5 μl of 50% acetonitrile each, allowed to dry and then 1 μl of CHCA was applied. At a third surface feature (spot #3) of the array, 1 μl of the peptide fragments was applied and allowed to dry. Spot #3 was washed three times with 5 μl of 50 mM ammonium acetate at pH 3.8, allowed to dry and then 1 μl CHCA was applied. At a fourth surface feature (spot #4) of the array, 1 μl of the peptide fragments was applied and allowed to dry. Spot #4 was washed three times with 5 μl of 50% acetonitrile, 0.1% TFA, allowed to cry and then 1 μl CHCA was applied.

2. Results

The peptide map of trypsin-digested transferrin detected for spot #1 of the H4 array was almost identical to the peptide map detected on gold array by MALDI. The peptide maps detected for spots #2 and #3 of the H4 array had fewer detected peptide fragments than the map detected on spot #1 since many were selectively washed away. Many peptide fragments that were retained on the H4 array through hydrophobic interaction on were washed away using the 50% acetonitrile solution. In addition, many negatively charged peptide fragments that were retained on the H4 array through ionic interaction were washed away using the 50 mM ammonium acetate, pH 3.8 buffer. Some peptide fragment peaks were newly detected or better detected on spots #2 and #3 of the H4 array thin on the gold array spot of the MALDI analysis. Further, there were very few detected peptide fragments on spot #4 after washing with the 50% acetonitiile, 0.1% TFA Solution. TIle combination of high organic solvent and low pH significantly reduced the association of the peptidc fragments with the C₁₈ groups on the H4 array. The results are discussed further with reference to accompanying figures as follows.

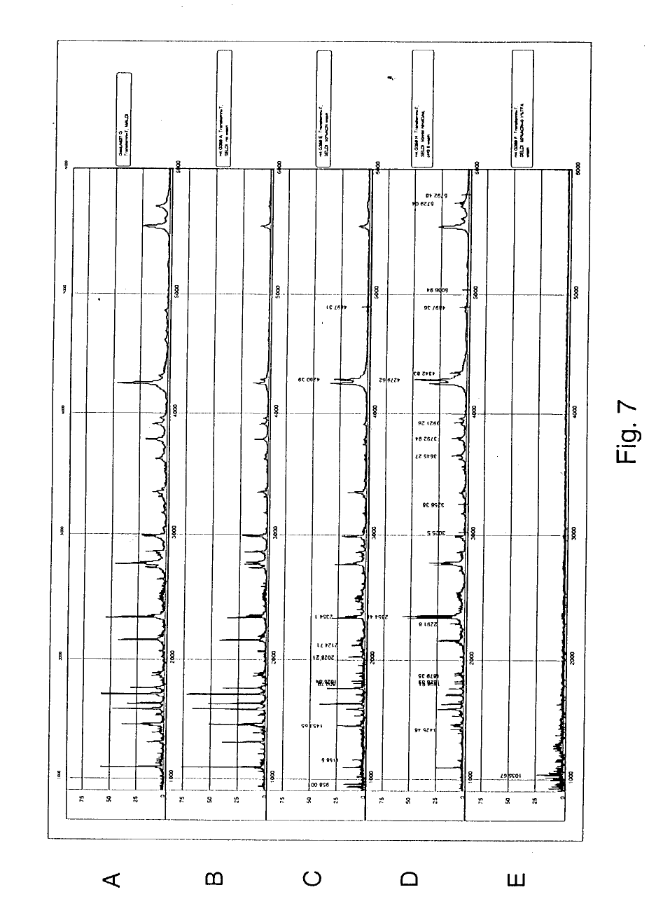

FIGS. 7A-E are mass spectral traces between 900 and 6000 Daltons (abscissa—Molecular Weight (Daltons); ordinate—relative intensity) showing the detection of peptide fragments from the typtic digest of the bovine transferrin described above. FIG. 7A shows a mass spectral trace obtained using MALDI on the gold array. FIG. 7B shows a mass spectral trace obtained using SELDI from the H4 array that involved no wash step prior to detection (i.e., spot #1). FIG. 7C shows a mass spectral trace obtained using SELDI from the H4 array that involved a 50% acetonitrile wash prior to detection (i.e., spot #2). FIG. 7D shows a mass spectral trace obtained using SELDI from the H4 array that involved the 50 nM ammonium acetate (pH 3.8) wash prior to detection (i.e., spot #3). FIG. 7E shows a mass spectral trace obtained using SELDI from the H4 array that involved the 50% acetonitrile, 0.1% TFA wash prior to detection (i.e., spot #4).

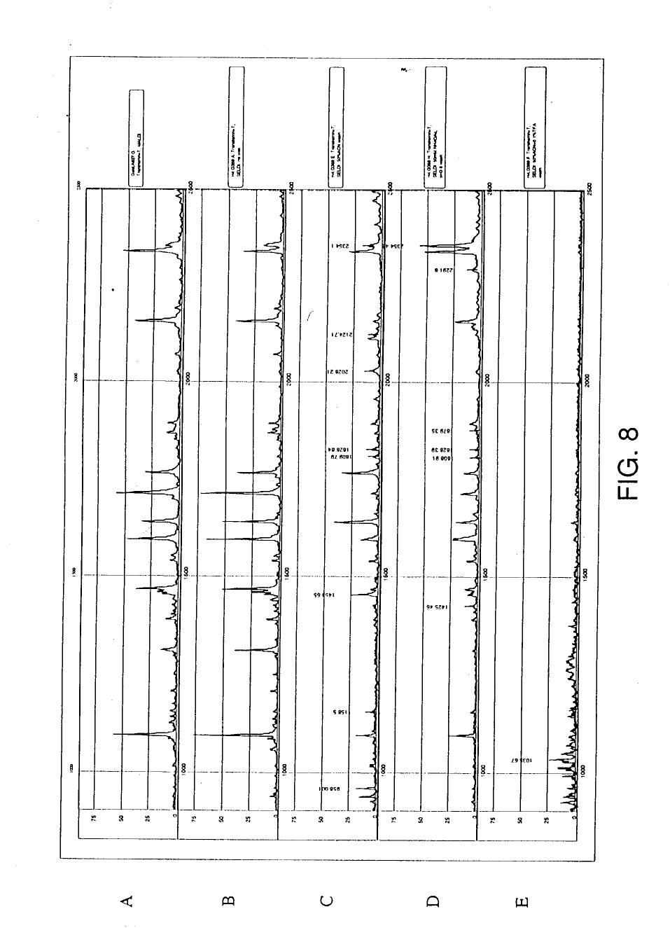

FIGS. 8A-E are mass spectral traces between 900 and 2500 Daltons (abscissa—Molecular Weight (Daltons); ordinate—relative intensity) showing the detection of peptide fragments from the tryptic digest of the bovine transferrin described above. FIG. 8A shows a mass spectral trace obtained using MALDI on the gold array. FIG. 8B shows a mass spectral trace obtained using SELDI from the H4 array that involved no wash step prior to detection (i.e., spot #1). FIG. 8C shows a mass spectral trace obtained using SELDI from the H4 array that involved the 50% acetonitrile wash prior to detection (i.e., spot #2). FIG. 8D shows a mass spectral trace obtained using SELDI from the H4 array that involved a 50 nM ammonium acetate (pH 3.8) wash prior to detection (i.e., spot #3). FIG. 8E shows a mass spectral trace obtained using SELDI from the H4 array that involved the 50% acetonitrile, 0.1% TFA wash prior to detection (i.e., spot #4). The labels indicate peaks that were detected better by SELDI than by MALDI.

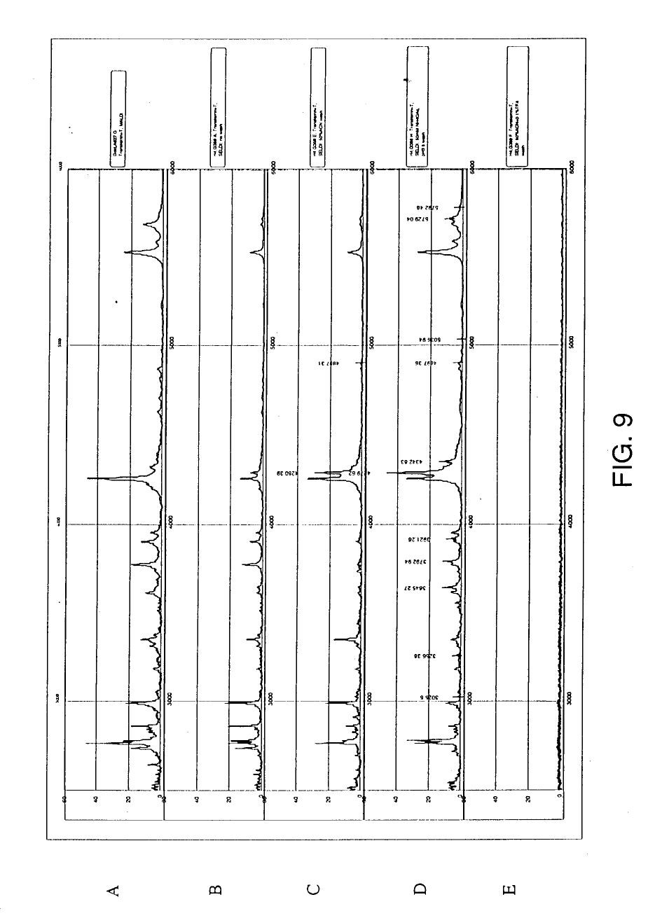

FIGS. 9A-E are mass spectral traces between 2500 and 6000 Daltons (abscissa—Molecular Weight (Daltons); originate—relative intensity) showing the detection of peptide fragments from the tryptic digest of the bovine transferrin described above. FIG. 9A shows a mass spectral trace obtained using MALDI on the gold array. FIG. 9B shows a mass spectral trace obtained using SELDI from the H4 array that involved no wash step prior to detection (i.e., spot #1). FIG. 9C shows a mass spectral trace obtained using SELDI from the H4 array that involved the 50% acetonitrile wash prior to detection (i.e., spot #2). FIG. 9D shows a mass spectral trace obtained using SELDI from the H4 array that involved the 50 nM ammonium acetate (pH 3.8) wash prior to detection (i.e., spot #3). FIG. 9E shows a mass spectral trace obtained using SELDI from the H4 array that involved the 50% acetonitrile, 0.1% TFA wash prior to detection (i.e., spot #4). The labels indicate peaks that were detected better by SELDI than by MALDI.

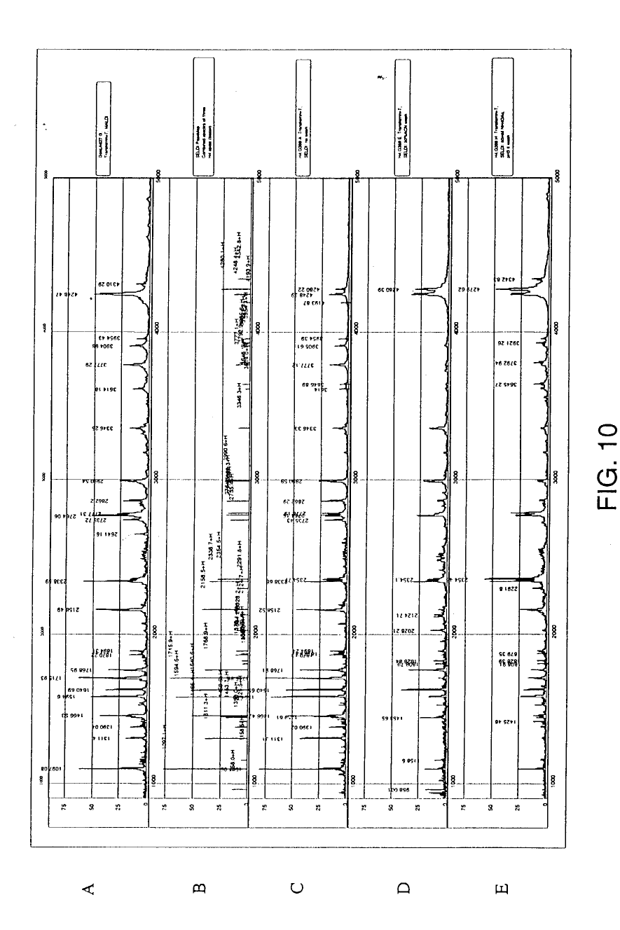

FIGS. 10A-E are mass spectral traces between 900 and 5000 Daltons (abscissa—Molecular—Weight (Daltons); ordinate—relative intensity) showing peptide maps of the tryptic digests of bovine transferrin described above. FIG. 10A shows a mass spectral trace obtained using MALDI on the gold array. FIG. 10B shows a combined mass spectral trace obtained using the SELDI data from three H4 array spots (i.e., spots #1-3). Each trace is shown separately in FIGS. 10C-E. In particular, FIG. 10C shows a mass spectral trace obtained using SELDI from the H4 array that involved no wash step prior to detection (i.e., spot #1). FIG. 10D shows a mass spectral trace obtained using SELDI from the H4 array that involved the 50% acetonitrile wash prior to detection (i.e., spot #2). FIG. 10E shows a mass spectral trace obtained using SELDI from the H4 array that involved the 50 mM ammonium acetate (pH 3.8) wash prior to detection (i.e., spot #4). The combined map obtained from the SELDI data shows more peptide fragment signals.

Following detection of peptide fragments as described above, a comparison of protein identification database searches using the MALDI and SELDI data was performed. Database searches were conducted using the ProFound and Mascot search engines. Protein identification for the peptide map generated by MALDI, and the “combined map” of 3 spectra from the H4 array (i.e., spots #1-3; see, FIG. 10) generated by SELDI showed the highest probable protein to be bovine transferrin. Both ProFound and Mascot search engines produced the same result. The confidence level, especially for Mascot's Mowse score, was higher for the SELDI data than for the MALDI data, because the number of detected peptide fragments that matched the calculated peptide fragments was greater. As for the ProFound search results, the next candidates after bovine the transferrin had much lower probabilities for the SELDI data as compared to the MALDI data.

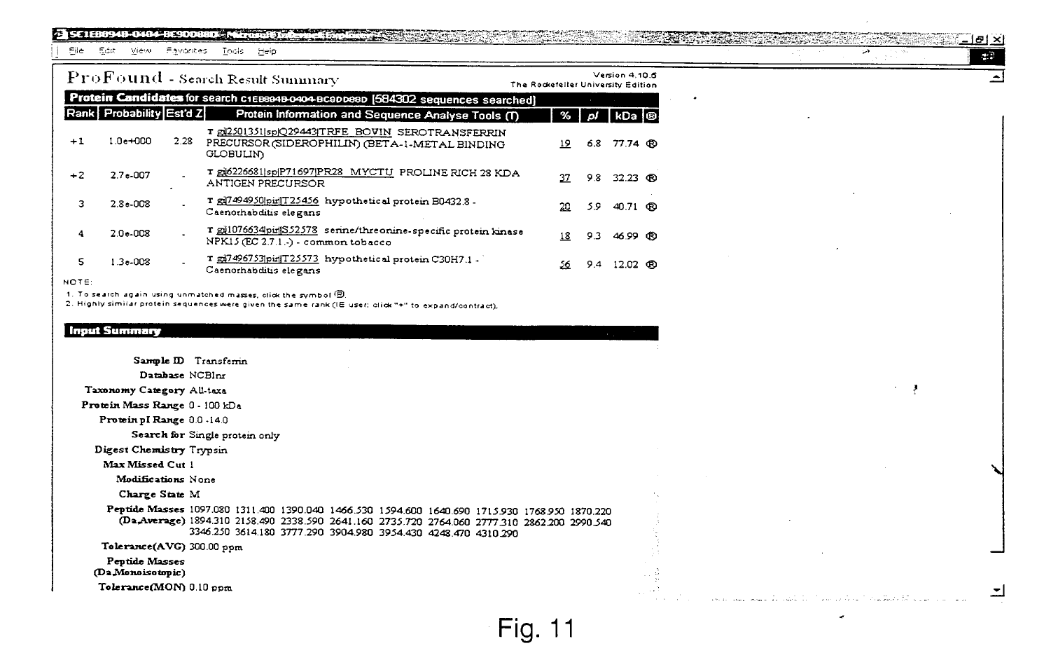

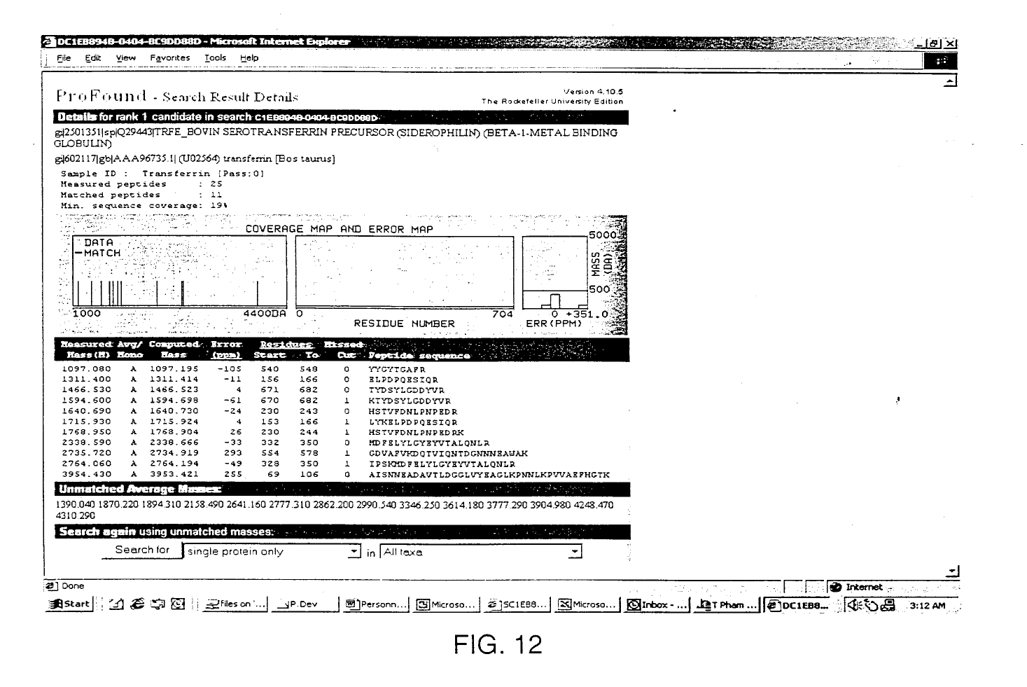

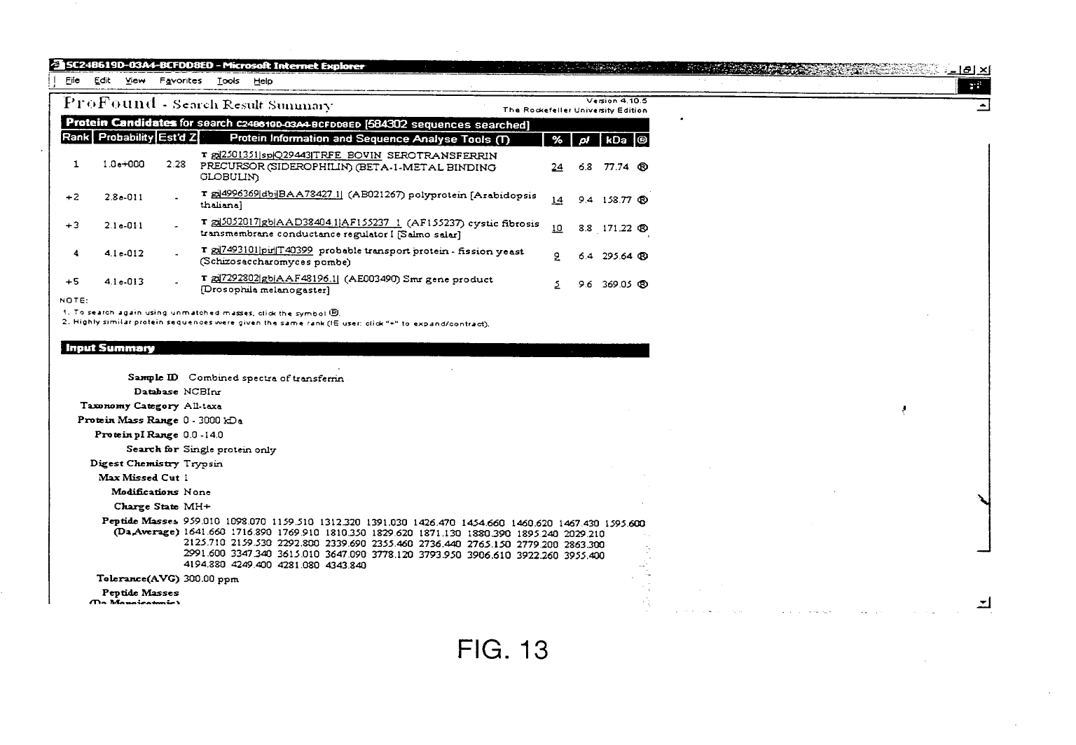

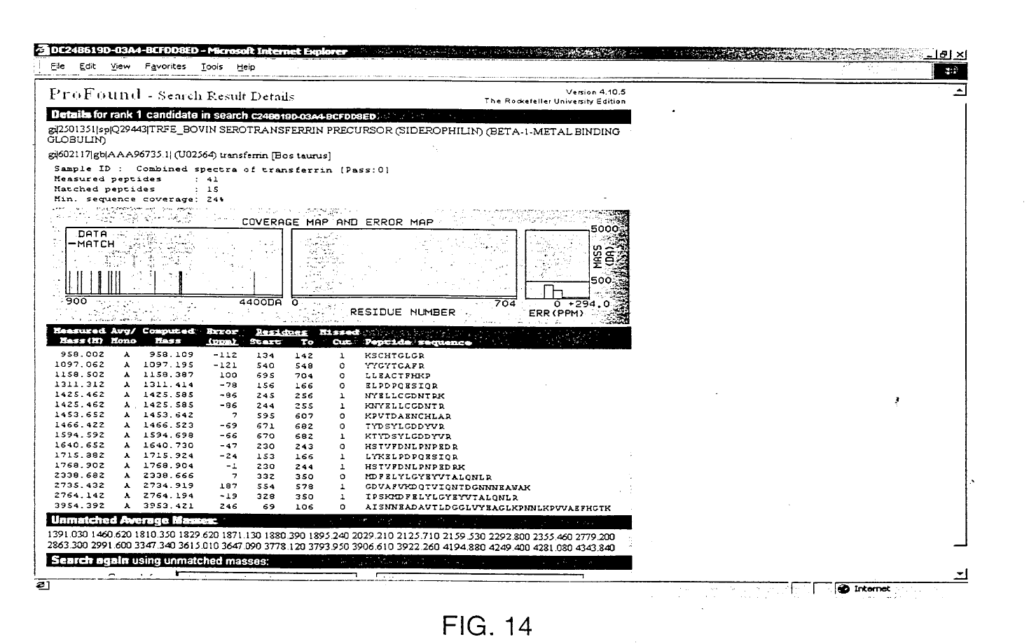

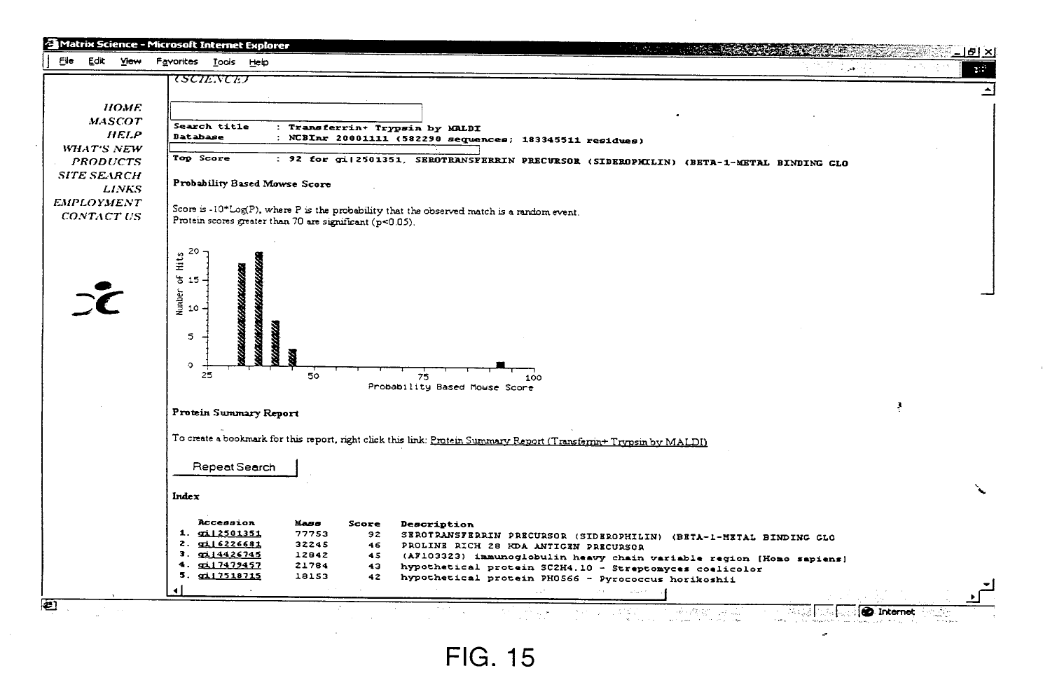

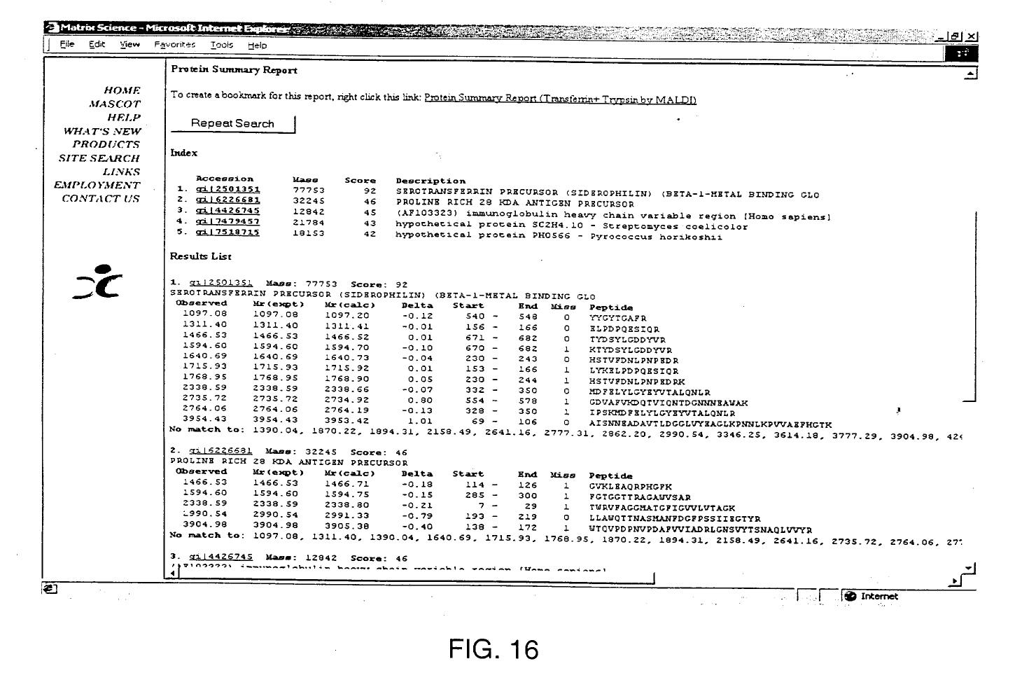

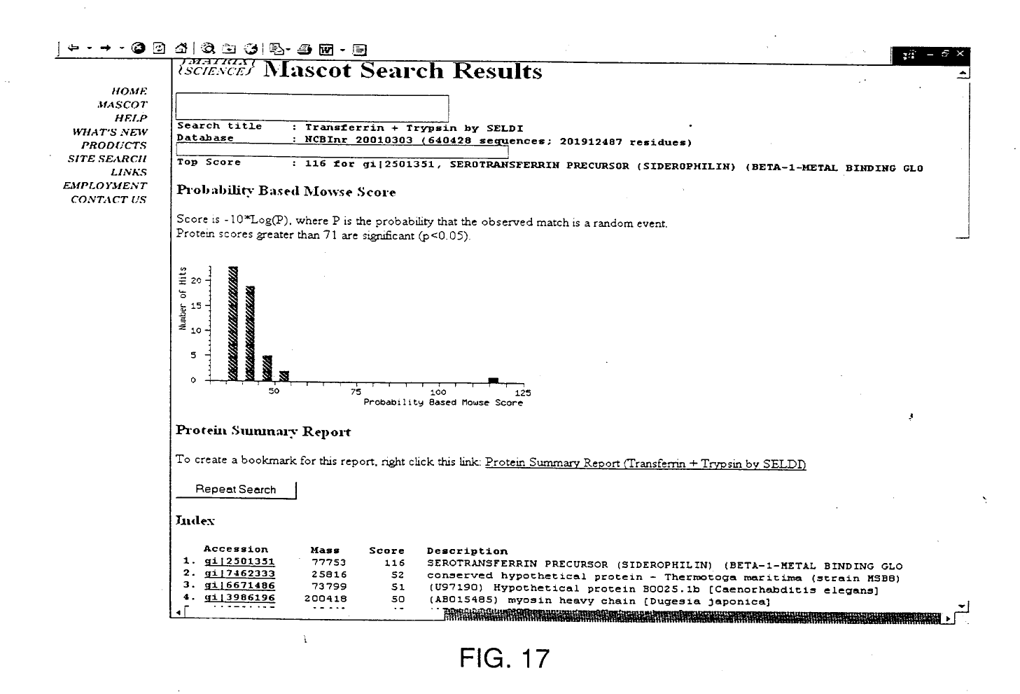

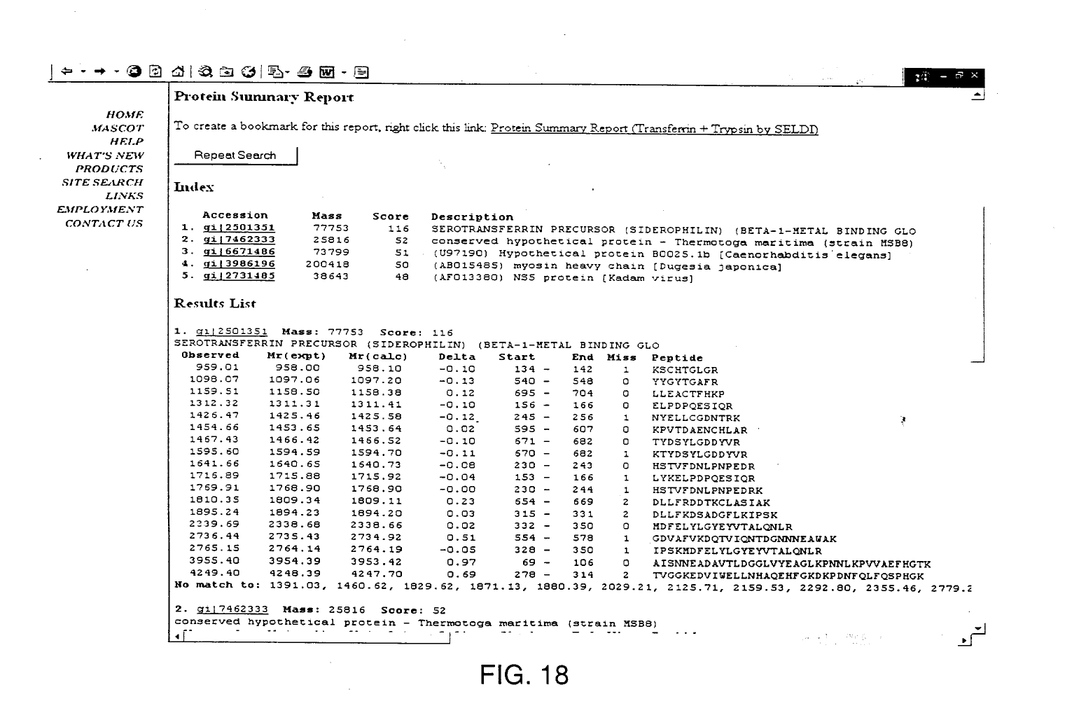

Figures showing display screens from the database searches are provided as follows. FIG. 11 shows a display screen for the ProFound database search using the peptide map generated by the MALDI analysis. FIG. 12 shows a display screen for the ProFound database search showing an analysis of the best candidate using the MALDI data. FIG. 13 shows a display screen for the ProFound database search using the peptide map generated by SELDI analysis. FIG. 14 shows a display screen for the ProFound database search showing an analysis of the best candidate using the SELDI data. FIG. 15 shows a display screen for the MASCOT database search using the peptide map generated by the MALDI analysis. FIG. 16 shows a display screen for the MASCOT database search showing an analysis of the best candidate using the MALDI data. FIG. 17 shows a display screen for the MASCOT database search using the peptide map generated by the SELDI analysis. FIG. 18 shows a display screen for the MASCOT database search showing an analysis of the best candidate using the SELDI data.

The present invention provides novel methods and systems for identifying target proteins. While specific examples halve been provided, the above description is illustrative and not restrictive. Any one or more of the features of the previously described embodiments call be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

All publications, patents, patent applications, or other documents cited in this application are incorporated by reference in their entirety for all purposes to the same extent as if each individual publication, patent, patent application, or other document were individually indicated to be incorporated by reference for all purposes. By their citation of various references in this document, Applicants do not admit any particular reference is “prior art” to their invention.

What is claimed is:

1. A method of producing at least one identity candidate for a target protein in a sample, comprising:
(a) fragmenting proteins in a first sample comprising the target protein to produce a fragmented sample comprising two or mole peptide fragments of the target protein;
(b) profiling peptide fragment masses in the fragmented sample by gas phase ion spectrometry under at least two different conditions,
wherein a first condition comprises analyzing a first aliquot of the fragmented sample by the gas phase ion spectrometry to produce a first set of peptide fragment mass data, and
wherein a second condition comprises fractionating biomolecules in a second aliquot of the fragmented sample by at least one first fractionation technique to produce at least one sub-sample comprising a peptide fragment of the target protein, and analyzing one or more sub-samples by the gas phase ion spectrometry to produce at least a second set of peptide fragment mass data; and,
(c) querying at least one database to produce the at least one identity candidate for the target protein based upon the first and second sets of peptide fragment mass data.

2. The method of claim 1, wherein the at least one identity candidate identifies the target protein.

3. The method of claim 1, wherein the target protein comprises at least about 50% by weight of total protein in the first sample.

4. The method of claim 1, wherein the target protein comprises at least about 50% of the total protein molecules in the first sample.

5. The method of claim 1, wherein the proteins in the first sample are fragmented enzymatically, chemically, or physically.

6. The method of claim 1, wherein the proteins in the first sample are fragmented by one or more proteases.

7. The method of claim 1, comprising producing identity candidates for multiple target proteins in the first sample.

8. The method of claim 1, further comprising generating a table of masses for peptide fragments in the first and second sets of peptide fragment mass data prior to (c).

9. The method of claim 1, further comprising comparing amounts of peptide fragments detected in the first or second sets of peptide fragment mass data with one or more controls.

10. The method of claim 1, wherein individual peptide fragments in the first or second sets of peptide fragment mass data are quantified.

11. The method of claim 1, wherein the at least one identity candidate for the target protein aids in the diagnosis of one or more pathological conditions.

12. The method of claim 1, further comprising fractionating biomolecules in an initial sample by one or more second fractionation techniques to collect an initial sample fraction that includes the target protein, wherein the initial sample fraction is used as the first sample in (a).

13. The method of claim 12, wherein the biomolecules in the initial sample are fractionated by:
(i) separating the biomolecules in the initial sample into a one- or two-dimensional array of spots, wherein each spot comprises one or more of the biomolecules; and
(ii) selecting and removing a spot from the array which is suspected of comprising the target protein.

14. The method of claims 1 or 12, wherein the one or more first or second fractionation techniques are independently selected from one or more of: electrophoresis, dialysis, filtration, or centrifugation.

15. The method of claims 1 or 12, wherein the one or more first or second fractionation techniques are independently selected from one or more of: affinity chromatography, high performance liquid chromatography, ion exchange chromatography, or size exclusion chromatography.

16. The method of claim 1, wherein the gas phase ion spectrometry comprises mass spectrometry.

17. The method of claim 16, wherein the mass spectrometry comprises laser desorption/ionization mass spectrometry.

18. The method of claim 17, wherein the laser desorption/ionization mass spectrometry is surface enhanced or matrix-assisted.

19. The method of claim 1, wherein gas phase ion spectrometeric analysis of the first aliquot comprises:
(i) contacting the first aliquot with at least one adsorbent bound to a surface of a probe which is removably insertable into a gas phase ion spectrometer; and
(ii) desorbing and ionizing peptide fragments in the first aliquot from the probe and detecting the desorbed/iodized peptide fragments with the gas phase ion spectrometer to provide the first set of peptide fragment mass data.

20. The method of claim 1, wherein gas phase ion spectrometeric analysis of the first aliquot comprises:
(i) contacting the first aliquot with at least one support-bound adsorbent;
(ii) placing the support-bound adsorbent on a probe, wherein the probe is removably insertable into a gas phase ion spectrometer; and
(iii) desorbing and ionizing peptide fragments in the first aliquot from the probe and detecting the desorbed/ionized peptide fragments with the gas phase ion spectrometer to provide the first set of peptide fragment mass data.

21. The method of claim 1, wherein gas phase ion spectrometeric analysis of the one or more sub-samples of the second aliquot comprises:
(i) contacting the second aliquot with the at least one adsorbent bound to a surface of a probe which is removably insertable into a gas phase ion spectrometer, wherein the at least one adsorbent captures one or more peptide fragments from the target protein;
(ii) removing non-captured material from the probe, wherein the one or more captured peptide fragments comprise a first sub-sample of the second aliquot; and
(iii) desorbing and ionizing the one or more captured peptide fragments from the probe and detecting the one or more desorbed/ionized peptide fragments with the gas phase ion spectrometer to provide the second set of peptide fragment mass data.

22. The method of claim 1, wherein gas phase ion spectrometeric analysis of the one or more sub-samples of the second aliquot comprises:
(i) contacting the second aliquot with at least one support-bound adsorbent, wherein the at least one support-bound adsorbent captures one or more peptide fragments from the target protein;
(ii) removing non-captured material from the at least one support-bound adsorbent, wherein the one or more captured peptide fragments on the at least one support-bound adsorbent comprise a first sub-sample of the second aliquot;
(iii) placing the at least one support-bound adsorbent on a probe, wherein the probe is removably insertable into a gas phase ion spectrometer; and
(iv) desorbing and ionizing the one or more captured peptide fragments from the probe and detecting the one or more desorbed/ionized peptide fragments with the gas phase ion spectrometer to provide the second set of peptide fragment mass data.

23. The method of claims 20 or 22, wherein the at least one support-bound adsorbent comprises a bead or resin derivatized with at least one adsorbent.

24. The method of claims 21 or 22, wherein the non-captured material is removed by one or more washes.

25. The method of claim 24, wherein each of the one or more washes comprises an identical or a different elution condition relative to at least one preceding wash.

26. The method of claim 25, wherein elution conditions differ according to pH, buffering capacity, ionic strength, a water structure characteristic, detergent type, detergent strength, hydrophobicity, dielectric constant, or concentration of at least one solute.

27. The method of claims 19, 20, 21, or 22, wherein the at least one adsorbent comprises at least one chromatographic adsorbent.

28. The method of claim 27, wherein the at least one chromatographic adsorbent comprises one or more of: an electrostatic adsorbent, a hydrophobic interaction adsorbent, a hydrophilic interaction adsorbent, a salt-promoted interaction adsorbent, a reversible covalent interaction adsorbent, or a coordinate covalent interaction adsorbent.

29. The method of claims 19, 20, 21, or 22, wherein the at least one adsorbent comprises at least one biomolecular interaction adsorbent.

30. The method of claim 29, wherein the at least one biomolecular interaction adsorbent comprises one or more of: all affinity adsorbent, a polypeptide, an enzyme, a receptor, or an antibody.

31. The method of claim 29, wherein the at least one biomolecular interaction adsorbent specifically captures at least one peptide fragment from the target protein.

32. The method of claims 19, 20, 21, or 22, wherein the probe comprises a substrate with at least one surface feature comprising the at least one adsorbent bound to the substrate, or capable of comprising the at least one support-bound adsorbent.

33. The method of claim 32, wherein the at least one adsorbent comprises at least one polypeptide that specifically binds an immunoglobulin and the method comprises exposing the first or second aliquot to the immunoglobulin, wherein the immunoglobulin specifically binds the one or more peptide fragments from the target protein, thereby forming a peptide fragment-complex, and contacting the peptide fragment-complex to the at least one adsorbent.

34. The method of claim 32, wherein the substrate comprises one or more of: glass, ceramic, plastic, a magnetic material, a polymer, an organic polymer, a conductive polymer, a native biopolymer, a metal, a metalloid, an alloy, or a metal coated with an organic polymer.

35. The method of claim 32, wherein the at least one surface feature comprises a plurality of surface features.

36. The method of claim 35, wherein the plurality of surface features is arranged in a line, an orthogonal array, a circle, or an n-sided polygon, wherein n is three or greater.

37. The method of claim 35, wherein the plurality of surface features comprises a logical or spatial array.

38. The method of claim 35, wherein each of the plurality of surface features comprises identical or different adsorbents, or one or more combinations thereof.

39. The method of claim 35, wherein at least two of the plurality of surface features comprise identical or different adsorbents, or one or more combinations thereof.

40. The method of claim 1, wherein the first and second sets of peptide fragment mass data are in a computer-readable form.

41. The method of claim 40, wherein (c) comprises operating a programmable computer and executing an algorithm that determines closeness-of-fit between the computer-readable data and database entries, which entries correspond to masses of identified proteins or peptide fragments therefrom, thereby producing the at least one identity candidate for the target protein based upon one or more detected peptide fragment masses in the first and second sets of peptide fragment mass data.

42. The method of claim 41, wherein the algorithm comprises an artificial intelligence algorithm or a heuristic learning algorithm.

43. The method of claim 42, wherein the artificial intelligence algorithm comprises one or more of: a fuzzy logic instruction set, a cluster analysis instruction set, a neural network, or a genetic algorithm.

44. A method of producing at least one identity candidate for a target protein, comprising:
(a) fragmenting proteins in a first sample comprising the target protein with one or more enzymes to produce a fragmented sample comprising two or more peptide fragments of the target protein;
(b) profiling peptide fragment masses in the fragmented sample by gas phase ion spectrometry under at least two different conditions,
wherein a first condition comprises analyzing a first aliquot of the fragmented sample by the gas phase ion spectrometry to produce a first set of peptide fragment mass data, and
wherein a second condition comprises fractionating biomolecules in a second aliquot of the fragmented sample by at least one first fractionation technique to produce at least one sub-sample comprising a peptide fragment of the target protein, and analyzing one or more sub-samples by the gas phase ion spectrometry to produce al least a second set of peptide fragment mass data; and,
(c) querying at least one database to produce the at least one identity candidate for the target protein based upon the first and second sets of peptide fragment mass data.

45. A method of producing at least one identity candidate for a target protein, comprising:
(a) fragmenting proteins in a first sample comprising the target protein with trypsin to produce a fragmented sample comprising two or more peptide fragments of the target protein;
(b) profiling peptide fragment masses in the fragmented sample by surface enhanced desorption/ionization time-of-flight mass spectrometry under at least two different conditions,
wherein a first condition comprises analyzing a first aliquot of the fragmented sample by the surface enhanced desorption/ionization time-of-flight mass spectrometry to produce a first set of peptide fragment mass data, and
wherein a second condition comprises fractionating biomolecules in a second aliquot of the fragmented sample by affinity chromatography to produce at least one sub-sample comprising a peptide fragment of the target protein, and analyzing one or more sub-samples by the surface enhanced desorption/ionization time-of-flight mass spectrometry to produce at least a second set of peptide fragment mass data; and,
(c) querying at least one database to produce the at least one identity candidate for the target protein based upon the first and second sets of peptide fragment mass data.

46. A system capable of producing at least one identity candidate for a target protein in a sample, comprising:
(a) one or more adsorbents capable of capturing peptide fragments in the sample under at least two different conditions;
(b) a gas phase ion spectrometer able to profile masses of peptide fragments captured by the one or more adsorbents tinder the at least two different conditions to provide at least two sets of peptide fragment mass data, each set corresponding to peptide fragments detected under a different condition; and,
(c) a processor, operably connected to the gas phase ion spectrometer, comprising at least one computer program providing logic instructions capable of determining closeness-of-fit between one or more detected peptide fragment masses in the sets of peptide fragment mass data and database entries, which entries correspond to masses of identified proteins or peptide fragments therefrom, thereby producing the at least one identity candidate for the target protein based upon the one or more detected peptide fragment masses.

47. The system of claim 46, wherein a computer comprises the processor and wherein the computer is external to the gas phase ion spectrometer.

48. The system of claim 46, wherein the one or more adsorbents comprise one or more solid phase adsorbents.

49. The system of claim 48, wherein the one or more solid phase adsorbents are provided as a probe comprising a substrate with at least one surface feature comprising the one or more solid phase adsorbents bound to the substrate.

50. The system of claim 49, wherein the probe is removably insertable into the gas phase ion spectrometer.

51. The system of claim 49, wherein the substrate comprises a plurality of surface features.

52. The system of claim 51, wherein the plurality of surface features is arranged in a line, an orthogonal array, a circle, or an n-sided polygon, wherein n is three or greater.

53. The system of claim 51, wherein the plurality of surface features comprises a logical or spatial array.

54. The system of claim 48, wherein the one or more solid phase adsorbents comprise beads or resins derivatized with the one of more adsorbents.

55. The system of claim 54, wherein the beads or resins derivatized with the one or more adsorbents are suitable for being placed on a probe removably insertable into the gas phase ion spectrometer.

56. The system of claim 46, wherein the gas phase ion spectrometer comprises the processor.

57. The system of claim 56, wherein the processor is a component of a computer.

58. The system of claim 46, wherein the gas phase ion spectrometer comprises a mass spectrometer.

59. The system of claim 58, wherein the mass spectrometer comprises a laser desorption/ionization mass spectrometer.
